(19) United States
(12) Patent Application Publication
(10) Pub. No.: US 2016/0002674 A1
(43) Pub. Date: Jan. 7, 2016

(54) METHOD FOR PRODUCING ETHANOL USING RECOMBINANT YEAST

(71) Applicant: TOYOTA JIDOSHA KABUSHIKI KAISHA, Toyota-shi, Aichi (JP)

(72) Inventors: Toru ONISHI, Toyota-shi, Aichi (JP); Nobuki TADA, Nisshin-shi, Aichi (JP); Noriko YASUTANI, Nagoya-shi, Aichi (JP); Satoshi KATAHIRA, Nagoya-shi, Aichi (JP); Nobuhiro ISHIDA, Seto-shi, Aichi (JP); Risa NAGURA, Toyota-shi, Aichi (JP)

(73) Assignee: TOYOTA JIDOSHA KABUSHIKI KAISHA, Toyota-shi, Aichi (JP)

(21) Appl. No.: 14/767,821

(22) PCT Filed: Feb. 27, 2014
(86) PCT No.: PCT/JP2014/054915

§ 371 Date: Aug. 13, 2015

(30) Foreign Application Priority Data
Feb. 27, 2013 (JP) 2013-037501
Feb. 27, 2014 (JP) 2014-036652

(51) Int. Cl. C12P 7/10

(52) U.S. Cl. CPC C12P 7/10

(57) ABSTRACT

The invention is intended to metabolize acetic acid and to lower acetic acid concentration in a medium at the time of xylose assimilation and ethanol fermentation by a yeast strain having xylose-metabolizing ability. The method for producing ethanol comprises a step of culturing recombinant yeast strains resulting from introduction of a xylose isomerase gene and an acetaldehyde dehydrogenase gene into a medium containing xylose, so as to perform ethanol fermentation.

### TECHNICAL FIELD

The present invention relates to a method for producing ethanol using a recombinant yeast strain having xylose-metabolizing ability.

### BACKGROUND ART

A cellulosic biomass is an effective starting material for a useful alcohol, such as ethanol, or an organic acid. In order to increase the amount of ethanol produced with the use of a cellulosic biomass, yeast strains capable of utilizing a xylose, which is a pentose, as a substrate have been developed. For example, Patent Literature 1 discloses a recombinant yeast strain resulting from incorporation of a xylose reductase gene and a xylitol dehydrogenase gene derived from Pichia stipitis and a xylulokinase gene derived from S. cerevisiae into its chromosome.

It is known that a large amount of acetic acid is contained in a hydrolysate of a cellulosic biomass and that acetic acid inhibits ethanol fermentation by a yeast strain. In the case of a yeast strain into which a xylose-assimilating gene has been introduced, in particular, acetic acid is known to inhibit ethanol fermentation carried out with the use of xylose as a saccharide source at a significant level (Non-Patent Literature 1 and 2).

A mash (moromi) resulting from fermentation of a cellulosic biomass saccharified with a cellulase is mainly composed of unfermented residue, poorly fermentable residue, enzymes, and fermenting microorganisms. Use of a mash-containing reaction solution for the subsequent fermentation process enables the reuse of fermenting microorganisms, reduction of the quantity of fermenting microorganisms to be introduced, and cost reduction. In such a case, however, acetic acid contained in the mash is simultaneously introduced, the concentration of acetic acid contained in a fermentation medium is increased as a consequence, and this may inhibit ethanol fermentation. In the case of a continuous fermentation technique in which the mash in a fermentation tank is transferred to a flash tank in which a reduced pressure level is maintained, ethanol is removed from the flash tank, and the mash is returned to the fermentation tank, although removal of acetic acid from the mash is difficult. Thus, inhibition of acetic acid-mediated fermentation would be critical.

In order to avoid inhibition of fermentation by acetic acid, there are reports concerning ethanol fermentation ability in the presence of acetic acid that has been improved by means of LPP1 or ENA1 gene overexpression (Non-Patent Literature 3) or FPS1 gene disruption (Non-Patent Literature 4) of Saccharomyces cerevisiae, which is a strain generally used for ethanol fermentation. However, such literature reports the results concerning ethanol fermentation conducted with the use of a glucose substrate, and the effects on ethanol fermentation conducted with the use of a xylose substrate, which is inhibited by acetic acid at a significant level, remain unknown. Even if the mutant yeast strains reported in such literature were used, the amount of acetic acid carry-over, which would be problematic at the time of the reuse of fermenting microorganisms or continuous fermentation, would not be reduced.

Alternatively, inhibition of fermentation by acetic acid may be avoided by metabolization of acetic acid in a medium simultaneously with ethanol fermentation. However, acetic acid metabolism is an aerobic reaction, which overlaps the metabolic pathway of ethanol. While acetic acid metabolism may be achieved by conducting fermentation under aerobic conditions, accordingly, ethanol as a target substance would also be metabolized.

As a means for metabolizing acetic acid under anaerobic conditions in which ethanol is not metabolized, assimilation of acetic acid achieved by introduction of the mhpF gene encoding acetaldehyde dehydrogenase (EC 1.2.1.10) into a Saccharomyces cerevisiae strain in which the GPD1 and GPD2 genes of the pathway of glycerine production had been destroyed has been reported (Non-Patent Literature 5 and Patent Literature 2). Acetaldehyde dehydrogenase catalyzes the reversible reaction described below.

Acetaldehyde+NAD⁺+coenzyme A [symbol] acetyl coenzyme A+NADH+H⁺

The pathway of glycerine production mediated by the GPD1 and GPD2 genes is a pathway that oxidizes excessive coenzyme NADH resulting from metabolism into NAD⁺, as shown in the following chemical reaction.

0.5 glucose+NADH+H⁺+ATP→glycerine+NAD⁺+ADP+Pi

The reaction pathway is destructed by disrupting the GPD1 and GPD2 genes, excessive coenzyme NADH is supplied through introduction of mhpF, and the reaction proceeds as shown below.

Acetyl coenzyme A+NADH+H⁺→acetaldehyde+NAD⁺+coenzyme A

Acetyl coenzyme A is synthesized from acetic acid by acetyl-CoA synthetase, and acetaldehyde is converted into ethanol. Eventually, excessive coenzyme NADH is oxidized and acetic acid is metabolized, as shown in the following chemical reaction.

Acetic acid+2NADH+2H⁺+ATP→ethanol+NAD⁺+AMP+Pi

As described above, it is necessary to destroy the glycerine pathway in order to impart acetic acid metabolizing ability to a yeast strain. However, the GPD1- and GPD2-disrupted strain is known to have significantly lowered fermentation ability, and utility at the industrial level is low. Neither Non-Patent Literature 5 nor Patent Literature 2 concerns the xylose-assimilating yeast strain, and, accordingly, whether or not the strain of interest would be effective at the time of xylose assimilation is unknown.

A strain resulting from introduction of the mhpF gene into a strain that was not subjected to GPD1 or GPD2 gene disruption has also been reported (Non-Patent Literature 6). While Non-Patent Literature 6 reports that the amount of acetic acid production is reduced upon introduction of the mhpF gene, it does not report that acetic acid in the medium would be reduced. In addition, Non-Patent Literature 6 does not relate to a xylose-assimilating yeast strain.

Also, there are reports concerning a xylose-assimilating yeast strain resulting from introduction of a xylose isomerase (XI) gene (derived from the intestinal protozoa of termites) (Patent Literature 3) and a strain resulting from further introduction of the acetaldehyde dehydrogenase gene (derived from Bifidobacterium adolescentis) into a xylose-assimilating yeast strain comprising a XI gene (derived from Piromyces sp. E2) introduced thereinto (Patent Literature 4), although the above literature does not report acetic acid assimilation at the time of xylose assimilation.

According to conventional techniques, as described above, acetic acid would not be efficiently metabolized or degraded under conditions in which ethanol fermentation and xylose assimilation take place simultaneously.

### CITATION LIST

### Patent Literature

### Patent Literature 1: JP 2009-195220 A

### Patent Literature 2: WO 2011/010923

### Patent Literature 3: JP 2011-147445 A

### Patent Literature 4: JP 2010-239925 A

### Non Patent Literature

### Non-Patent Literature 1: FEMS Yeast Research, vol. 9, 2009, pp. 358-364

### Non-Patent Literature 2: Enzyme and Microbial Technology 33, 2003, pp. 786-792

Non-Patent Literature 3: Biotechnol. Bioeng., 2009, 103 (3): pp. 500-512
Non-Patent Literature 4: Biotechnol. Lett., 2011, 33: pp. 277-284
Non-Patent Literature 5: Appl. Environ. Microbiol., 2010, 76: pp. 190-195
Non-Patent Literature 6: Biotechnol. Lett., 2011, 33: pp. 1375-1380

### SUMMARY OF THE INVENTION

### Technical Problem

Under the above circumstances, it is an object of the present invention to provide a method for producing ethanol using a recombinant yeast strain capable of metabolizing acetic acid in a medium to lower acetic acid concentration therein when performing xylose assimilation and ethanol fermentation using a yeast strain having xylose-metabolizing ability, so as to improve ethanol productivity.

### Solution to Problem

The present inventors have conducted concentrated studies in order to attain the above object. As a result, they discovered that a recombinant yeast strain resulting from introduction of a particular acetaldehyde dehydrogenase gene into a yeast strain having xylose-metabolizing ability would enable metabolization of acetic acid in a medium when performing ethanol fermentation in a xylose-containing medium. This has led to the completion of the present invention.

The present invention includes the following.

(1) A method for producing ethanol comprising steps of culturing a recombinant yeast strain comprising a xylose isomerase gene and an acetaldehyde dehydrogenase gene introduced thereinto in a xylose-containing medium to perform ethanol fermentation.
(2) The method for producing ethanol according to (1), wherein the xylose isomerase gene encodes the protein (a) or (b) below:
(a) a protein comprising the amino acid sequence as shown in SEQ ID NO: 4; or
(b) a protein comprising an amino acid sequence having 70% or higher identity with the amino acid sequence as shown in SEQ ID NO: 4 and having enzyme activity of converting xylose into xylulose.
(3) The method for producing ethanol according to (1), wherein the acetaldehyde dehydrogenase gene encodes acetaldehyde dehydrogenase derived from E. coli.
(4) The method for producing ethanol according to (3), wherein the acetaldehyde dehydrogenase derived from E. coli is the protein (a) or (b) below:
(a) a protein comprising the amino acid sequence as shown in SEQ ID NO: 2 or 20; or
(b) a protein comprising an amino acid sequence having 70% or higher identity with the amino acid sequence as shown in SEQ ID NO: 2 or 20 and having acetaldehyde dehydrogenase activity.
(5) The method for producing ethanol according to (1), wherein the acetaldehyde dehydrogenase gene encodes acetaldehyde dehydrogenase derived from Clostridium beijerinckii.
(6) The method for producing ethanol according to (5), wherein the acetaldehyde dehydrogenase derived from Clostridium beijerinckii is the protein (a) or (b) below:
(a) a protein comprising the amino acid sequence as shown in SEQ ID NO: 22; or
(b) a protein comprising an amino acid sequence having 70% or higher identity with the amino acid sequence as shown in SEQ ID NO: 22 and having acetaldehyde dehydrogenase activity.
   (7) The method for producing ethanol according to (1), wherein the acetaldehyde dehydrogenase gene encodes acetaldehyde dehydrogenase derived from Chlamydomonas reinhardtii.
   (8) The method for producing ethanol according to (5), wherein the acetaldehyde dehydrogenase derived from Chlamydomonas reinhardtii is the protein (a) or (b) below:
   (a) a protein comprising the amino acid sequence as shown in SEQ ID NO: 24; or
   (b) a protein comprising an amino acid sequence having 70% or higher identity with the amino acid sequence as shown in SEQ ID NO: 24 and having acetaldehyde dehydrogenase activity.
   (9) The method for producing ethanol according to (1), wherein the recombinant yeast strain further comprises the xylulokinase gene introduced thereinto.
   (10) The method for producing ethanol according to (1), wherein the recombinant yeast strain comprises a gene encoding an enzyme selected from the group of enzymes constituting a non-oxidative process in the pentose phosphate pathway.
   (11) The method for producing ethanol according to (10), wherein the group of enzymes constituting a non-oxidative process in the pentose phosphate pathway includes ribose-5-phosphate isomerase, ribulose-5-phosphate-3-epimerase, transketolase, and transaldolase.
   (12) The method for producing ethanol according to (1), wherein the medium contains cellulose and the ethanol fermentation proceeds simultaneously with saccharification by at least the cellulose.
   (13) The method for producing ethanol according to (1), wherein the recombinant yeast strain allows high-level expression of the alcohol dehydrogenase gene having activity of converting acetaldehyde into ethanol.
   (14) The method for producing ethanol according to (1), wherein the recombinant yeast strain shows a lowered expression level of the alcohol dehydrogenase gene having activity of converting ethanol into acetaldehyde.

The present application claims priority from Japanese patent applications JP 2013-037501 and JP 2014-36652, the contents of which are hereby incorporated by reference into this application.

### Advantageous Effects of Invention

According to the method for producing ethanol of the present invention, acetic acid concentration in a medium can be lowered, and inhibition of fermentation caused by acetic acid can be effectively avoided. As a result, the method for producing ethanol of the present invention is capable of maintaining high efficiency for ethanol fermentation performed with the use of xylose as a saccharide source and achieving excellent ethanol yield. Accordingly, the method for producing ethanol of the present invention enables reduction of the amount of acetic acid carry-over at the time of, for example, the reuse of the recombinant yeast strain or use thereof for continuous culture, thereby allowing maintenance of an excellent ethanol yield.

### BRIEF DESCRIPTION OF DRAWINGS

FIG. 1 schematically shows a constitution of pUC-HIS3U-P_HOR7-XKS1-T_TDH3-P_TDH2-hph-T_CYC1-HIS3D.

FIG. 2 schematically shows a constitution of pUC-R67-HOR7p-RsXI-T_TDH3-TRP1d-R45.

FIG. 3 schematically shows a constitution of pUC-LEU2U-P_HOR7-TAL1-T_TDH3-P_HOR7-TKL1-T_TDH3-HIS3-LEU2 D.

FIG. 4 schematically shows a constitution of pUC-GRE3U-P_HOR7-RPE1-T_TDH3-P_HOR7-RKI1-T_TDH3-LEU2-GRE3 D.

FIG. 5 schematically shows a constitution of pCR-ADH2U-URA3-ADH2D.

FIG. 6 schematically shows a constitution of pCR-ADH2part-T_CYC1-P_TDH3-ADH1-T_ADH1-URA3-ADH2D.

FIG. 7 schematically shows a constitution of pCR-ADH2part-T_CYC1-ERO1_T-mhpF-HOR7_P-URA3-ADH2D.

FIG. 8 schematically shows a constitution of pCR-ADH2part-T_CYC1-P_TDH3-ADH1-T_ADH1-ERO1_T-mhpF-HOR7_P-URA3-ADH2D.

FIG. 9 schematically shows a constitution of pCR-ADH2U-ERO1_T-mhpF-HOR7_P-URA3-ADH2D.

FIG. 10 schematically shows a constitution of pCR-ADH2U-P_TDH3-ADH1-T_ADH1-ERO1_T-mhpF-HOR7_P-URA3-ADH 2D.

FIG. 11 schematically shows a constitution of pCR-ADH2part-T_CYC1-URA3-ADH2D.

### DESCRIPTION OF EMBODIMENTS

Hereafter, the present invention is described in greater detail with reference to the drawings and the examples.

The method for producing ethanol of the present invention is a method for synthesizing ethanol from a saccharide source contained in a medium with the use of a recombinant yeast strain having xylose-metabolizing ability into which an acetaldehyde dehydrogenase gene has been introduced. According to the method for producing ethanol of the present invention, since the recombinant yeast strain can metabolize acetic acid contained in a medium, acetic acid concentration in a medium is lowered in association with ethanol fermentation.

### <Recombinant Yeast Strain>

A recombinant yeast strain used in the method for producing ethanol of the present invention comprises the xylose isomerase gene and the acetaldehyde dehydrogenase gene introduced thereinto, which is a yeast strain having xylose-metabolizing ability. The term “yeast strain having xylose-metabolizing ability” refers to any of the following: a yeast strain to which xylose-metabolizing ability has been imparted as a result of introduction of a xylose isomerase gene into a yeast strain that does not inherently has xylose-metabolizing ability; a yeast strain to which xylose-metabolizing ability has been imparted as a result of introduction of a xylose isomerase gene and another xylose metabolism-associated gene into a yeast strain that does not inherently have xylose-metabolizing ability; and a yeast strain that inherently has xylose-metabolizing ability.

A yeast strain having xylose-metabolizing ability is capable of assimilating xylose contained in a medium to produce ethanol. Xylose contained in a medium may be obtained by saccharification of xylan or hemicellulose comprising xylose as a constituent sugar. Alternatively, it may be supplied to a medium as a result of saccharification of xylan or hemicellulose contained in a medium by a saccharification-enzyme. In the case of the latter, the term “xylose contained in a medium” refers to the so-called simultaneous saccharification and fermentation process.

The xylose isomerase gene (the XI gene) is not particularly limited, and a gene originating from any organism species may be used. For example, a plurality of the xylose isomerase genes derived from the intestinal protozoa of termites disclosed in JP 2011-147445 A can be used without particular limitation. Examples of the xylose isomerase genes that can be used include a gene derived from the anaerobic fungus Piromyces sp. strain E2 (JP 2005-514951 A), a gene derived from the anaerobic fungus Cyllamyces aberensis, a gene derived from another bacterial strain (i.e., Bacteroides thetaiotaomicron), a gene derived from a bacterial strain (i.e., Clostridium phytofermentans), and a gene derived from the Streptomyces murinus cluster.

Specifically, use of a xylose isomerase gene derived from the intestinal protozoa of Reticulitermes speratus as the xylose isomerase gene is preferable. The nucleotide sequence of the coding region of the xylose isomerase gene derived from the intestinal protozoa of Reticulitermes speratus and the amino acid sequence of a protein encoded by such gene are shown in SEQ ID NOs: 3 and 4, respectively.

The xylose isomerase genes are not limited to the genes identified by SEQ ID NOs: 3 and 4. It may be a paralogous gene or a homologous gene in the narrow sense having different nucleotide and amino acid sequences.

The xylose isomerase genes are not limited to the genes identified by SEQ ID NOs: 3 and 4. For example, it may be a gene comprising an amino acid sequence having 70% or higher, preferably 80% or higher, more preferably 90% or higher, and most preferably 95% or higher sequence similarity to or identity with the amino acid sequence as shown in SEQ ID NO: 4 and encoding a protein having xylose isomerase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by such amino acid residues.

Further, the xylose isomerase genes are not limited to the genes identified by SEQ ID NOs: 3 and 4. For example, it may be a gene comprising an amino acid sequence derived from the amino acid sequence as shown in SEQ ID NO: 4 by substitution, deletion, insertion, or addition of one or several amino acids and encoding a protein having acetaldehyde dehydrogenase activity. The term “several” used herein refers to, for example, 2 to 30, preferably 2 to 20, more preferably 2 to 10, and most preferably 2 to 5.

Furthermore, the xylose isomerase genes are not limited to the genes identified by SEQ ID NOs: 3 and 4. For example, it may be a gene hybridizing under stringent conditions to the full-length sequence or a partial sequence of a complementary strand of DNA comprising the nucleotide sequence as shown in SEQ ID NO: 3 and encoding a protein having xylose isomerase activity. Under “stringent conditions,” so-called specific hybrids are formed, but non-specific hybrids are not formed. Such conditions can be adequately determined with reference to, for example, Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42° C. to 68° C. and preferably 42° C. to 65° C., for example. Further specifically, the sodium concentration is 5×SSC (83 mM NaCl, 83 mM sodium citrate), and the temperature is 42° C.

As described above, whether or not a gene comprising a nucleotide sequence that differs from the sequence shown in SEQ ID NO: 3 or a gene encoding an amino acid sequence that differs from the sequence shown in SEQ ID NO: 4 would function as a xylose isomerase gene may be determined by, for example, preparing an expression vector comprising the gene of interest incorporated into an adequate site between a promoter and a terminator, transforming an E. coli host using such expression vector, and assaying the xylose isomerase activity of the protein expressed. The term “xylose isomerase activity” refers to activity of isomerizing xylose into xylulose. Accordingly, xylose isomerase activity can be evaluated by preparing a xylose-containing solution as a substrate, allowing the target protein to react at an adequate temperature, and measuring the amount of xylose that has decreased and/or the amount of xylulose that has been generated.

It is particularly preferable to use, as a xylose isomerase gene, a gene encoding mutated xylose isomerase comprising the amino acid sequence as shown in SEQ ID NO: 4 having a specific mutation of a particular amino acid residue and thus having improved xylose isomerase activity. A specific example of a gene encoding mutated xylose isomerase is a gene encoding the amino acid sequence as shown in SEQ ID NO: 4 in which asparagine at amino acid position 337 has been substituted with cysteine. Xylose isomerase comprising the amino acid sequence as shown in SEQ ID NO: 4 in which asparagine at amino acid position 337 has been substituted with cysteine has xylose isomerase activity superior to that of wild-type xylose isomerase. In addition, mutated xylose isomerase is not limited to xylose isomerase in which asparagine at amino acid position 337 has been substituted with cysteine. It may be xylose isomerase in which asparagine at amino acid position 337 has been substituted with a different amino acid other than cysteine, xylose isomerase in which asparagine at amino acid position 337 has been substituted with a different amino acid and further substitution of a different amino acid residue has taken place, or xylose isomerase in which an amino acid residue other than asparagine at amino acid position 337 has been substituted with a different amino acid.

Meanwhile, examples of xylose metabolism-associated genes other than the xylose isomerase gene include a xylose reductase gene encoding a xylose reductase that converts xylose into xylitol, a xylitol dehydrogenase gene encoding a xylitol dehydrogenase that converts xylitol into xylulose, and a xylulokinase gene encoding a xylulokinase that phosphorylates xylulose to produce xylulose 5-phosphate. Xylulose 5-phosphate produced by a xylulokinase enters the pentose phosphate pathway, and it is then metabolized therein.

Examples of xylose metabolism-associated genes include, but are not particularly limited to, a xylose reductase gene and a xylitol dehydrogenase gene derived from Pichia stipitis and a xylulokinase gene derived from Saccharomyces cerevisiae (see Eliasson A. et al., Appl. Environ. Microbiol., 66: 3381-3386; and Toivari M. N. et al., Metab. Eng., 3: 236-249). In addition, xylose reductase genes derived from Candida tropicalis and Candida prapsilosis, xylitol dehydrogenase genes derived from Candida tropicalis and Candida prapsilosis, and a xylulokinase gene derived from Pichia stipitis can be used.

Examples of yeast strains that inherently have xylose-metabolizing ability include, but are not particularly limited to, Pichia stipitis, Candida tropicalis, and Candida prapsilosis.

An acetaldehyde dehydrogenase gene to be introduced into a yeast strain having xylose-metabolizing ability is not particularly limited, and a gene derived from any species of organism may be used. When acetaldehyde dehydrogenase genes derived from organisms other than a fungus such as yeast (e.g., genes derived from bacteria, animals, plants, insects, or algae) are used, it is preferable that the nucleotide sequence of the gene be modified in accordance with the frequency of codon usage in a yeast strain into which the gene of interest is to be introduced.

More specifically, the mhpF gene of E. coli or the ALDH1 gene of Entamoeba histolytica as disclosed in Applied and Environmental Microbiology, May 2004, pp. 2892-2897, Vol. 70, No. 5 can be used as the acetaldehyde dehydrogenase genes. The nucleotide sequence of the mhpF gene of E. coli and the amino acid sequence of a protein encoded by the mhpF gene are shown in SEQ ID NOs: 1 and 2, respectively.

The acetaldehyde dehydrogenase genes are not limited to the genes identified by SEQ ID NOs: 1 and 2. It may be a paralogous gene or a homologous gene in the narrow sense having different nucleotide and amino acid sequences as long as it encodes an enzyme defined with EC No. 1.2.1.10. Examples of the acetaldehyde dehydrogenase genes include an adhE gene of E. coli, an acetaldehyde dehydrogenase gene derived from Clostridium beijerinckii, and an acetaldehyde dehydrogenase gene derived from Chlamydomonas reinhardtii. Here, the nucleotide sequence of the adhE gene of E. coli and the amino acid sequence of a protein encoded by the adhE gene are shown in SEQ ID NOs: 19 and 20, respectively. In addition, the nucleotide sequence of the acetaldehyde dehydrogenase gene derived from Clostridium beijerinckii and the amino acid sequence of a protein encoded by the gene are shown in SEQ ID NOs: 21 and 22, respectively. Further, the nucleotide sequence of the acetaldehyde dehydrogenase gene derived from Chlamydomonas reinhardtii and the amino acid sequence of a protein encoded by the gene are shown in SEQ ID NOs: 23 and 24, respectively.

The acetaldehyde dehydrogenase genes are not limited to the genes identified by SEQ ID NOs: 1 and 2, 19 and 20, 21 and 22, or 23 and 24. For example, it may be a gene comprising an amino acid sequence having 70% or higher, preferably 80% or higher, more preferably 90% or higher, and most preferably 95% or higher sequence similarity to or identity with the amino acid sequence as shown in SEQ ID NO: 2, 20, 22, or 24 and encoding a protein having acetaldehyde dehydrogenase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of the aforementioned amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by such completely identical amino acid residues.

Further, the acetaldehyde dehydrogenase genes are not limited to the genes identified by SEQ ID NOs: 1 and 2, 19 and 20, 21 and 22, or 23 and 24. For example, it may be a gene comprising an amino acid sequence derived from the amino acid sequence as shown in SEQ ID NO: 2, 20, 22, or 24 by substitution, deletion, insertion, or addition of one or several amino acids and encoding a protein having acetaldehyde dehydrogenase activity. The term “several” used herein refers to, for example, 2 to 30, preferably 2 to 20, more preferably 2 to 10, and most preferably 2 to 5.

Furthermore, the acetaldehyde dehydrogenase genes are not limited to the genes identified by SEQ ID NOs: 1 and 2, 19 and 20, 21 and 22, or 23 and 24. For example, it may be a gene hybridizing under stringent conditions to the full-length sequence or a partial sequence of a complementary strand of DNA comprising the nucleotide sequence as shown in SEQ ID NO: 1, 19, 21, or 23 and encoding a protein having acetaldehyde dehydrogenase activity. Under “stringent conditions,” so-called specific hybrids are formed, but non-specific hybrids are not formed. Such conditions can be adequately determined with reference to, for example, Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42° C. to 68° C. and preferably 42° C. to 65° C., for example. Further specifically, the sodium concentration is 5×SSC (83 mM NaCl, 83 mM sodium citrate), and the temperature is 42° C.

As described above, whether or not a gene comprising a nucleotide sequence that differs from the sequence shown in SEQ ID NO: 1, 19, 21, or 23 or a gene encoding an amino acid sequence that differs from the sequence shown in SEQ ID NO: 2, 20, 22, or 24 would function as an acetaldehyde dehydrogenase gene may be determined by, for example, preparing an expression vector comprising the gene of interest incorporated into an adequate site between a promoter and a terminator, transforming an E. coli host using such expression vector, and assaying acetaldehyde dehydrogenase activity of the protein expressed. Acetaldehyde dehydrogenase activity can be assayed by preparing a solution containing acetaldehyde, CoA, and NAD⁺ as substrates, allowing the target protein to react at adequate temperature, and converting the generated acetyl phosphate into acetyl phosphate with the aid of a phosphate acetyl transferase or spectroscopically assaying the generated NADH.

A recombinant yeast strain used in the method for producing ethanol of the present invention has xylose-metabolizing ability and comprises at least the acetaldehyde dehydrogenase gene introduced thereinto. A recombinant yeast strain may further comprise other gene(s) introduced thereinto, and such other gene(s) are not particularly limited. For example, a gene involved in the sugar metabolism of glucose may be introduced into such recombinant yeast strain. For example, a recombinant yeast strain can have β-glucosidase activity resulting from the introduction of the β-glucosidase gene.

The term “β-glucosidase activity” used herein refers to the activity of catalyzing a hydrolysis reaction of a β-glycoside bond of a sugar. Specifically, β-glucosidase is capable of degrading a cellooligosaccharide, such as cellobiose, into glucose. The β-glucosidase gene can be introduced in the form of a cell-surface display gene. The term “cell-surface display gene” used herein refers to a gene that is modified to display a protein to be encoded by the gene on a cell surface. For example, a cell-surface display β-glucosidase gene is a gene resulting from fusion of a β-glucosidase gene with a cell-surface localized protein gene. A cell-surface localized protein is fixed and present on a yeast cell surface layer. Examples include agglutinative proteins, such as α- or a-agglutinin and FLO proteins. In general, a cell-surface localized protein comprises an N-terminal secretory signal sequence and a C-terminal GPI anchor attachment recognition signal. While a cell-surface localized protein shares properties with a secretory protein in terms of the presence of a secretory signal, its secretory signal differs in that the cell-surface localized protein is transported while fixed to a cell membrane through a GPI anchor. When a cell-surface localized protein passes through a cell membrane, a GPI anchor attachment recognition signal sequence is selectively cut, it binds to a GPI anchor at a newly protruded C-terminal region, and it is then fixed to the cell membrane. Thereafter, the root of the GPI anchor is cut by phosphatidylinositol-dependent phospholipase C (PI-PLC). Subsequently, a protein separated from the cell membrane is integrated into a cell wall, fixed onto a cell surface layer, and then localized on a cell surface layer (see, for example, JP 2006-174767 A).

The β-glucosidase gene is not particularly limited, and an example is a β-glucosidase gene derived from Aspergillus aculeatus (Murai, et al., Appl. Environ. Microbiol., 64: 4857-4861). In addition, a β-glucosidase gene derived from Aspergillus oryzae, a β-glucosidase gene derived from Clostridium cellulovorans, and a β-glucosidase gene derived from Saccharomycopsis fibligera can be used.

In addition to or other than the β-glucosidase gene, a gene encoding another cellulase-constituting enzyme may have been introduced into a recombinant yeast strain used in the method for producing ethanol of the present invention. Examples of cellulase-constituting enzymes other than β-glucosidase include exo-cellobiohydrolases that liberate cellobiose from the terminus of crystalline cellulose (CBH1 and CBH2) and endo-glucanase (EG) that cannot degrade crystalline cellulose but cleaves a non-crystalline cellulose (amorphous cellulose) chain at random.

Examples of other genes to be introduced into a recombinant yeast strain include an alcohol dehydrogenase gene (the ADH1 gene) having activity of converting acetaldehyde into ethanol, an acetyl-CoA synthetase gene (the ACS1 gene) having activity of converting acetic acid into acetyl-CoA, and genes having activity of converting acetaldehyde into acetic acid (i.e., the ALD4, ALD5, and ALD6 genes). The alcohol dehydrogenase gene (the ADH2 gene) having activity of converting ethanol into acetaldehyde may be disrupted.

In addition, it is preferable that a recombinant yeast strain used in the method for producing ethanol of the present invention allow high-level expression of the alcohol dehydrogenase gene (the ADH1 gene) having activity of converting acetaldehyde into ethanol. In order to realize high-level expression of such gene, for example, a promoter of the inherent gene may be replaced with a promoter intended for high-level expression, or an expression vector enabling expression of such gene may be introduced into a yeast strain.

The nucleotide sequence of the ADH1 gene of Saccharomyces cerevisiae and the amino acid sequence of a protein encoded by such gene are shown in SEQ ID NOs: 5 and 6, respectively. The alcohol dehydrogenase gene to be expressed at high level is not limited to the genes identified by SEQ ID NOs: 5 and 6. It may be a paralogous gene or a homologous gene in the narrow sense having different nucleotide and amino acid sequences.

The alcohol dehydrogenase genes are not limited to the genes identified by SEQ ID NOs: 5 and 6. For example, it may be a gene comprising an amino acid sequence having 70% or higher, preferably 80% or higher, more preferably 90% or higher, and most preferably 95% or higher sequence similarity to or identity with the amino acid sequence as shown in SEQ ID NO: 6 and encoding a protein having alcohol dehydrogenase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by the total number of such amino acid residues.

Further, the alcohol dehydrogenase genes are not limited to the genes identified by SEQ ID NOs: 5 and 6. For example, it may be a gene comprising an amino acid sequence derived from the amino acid sequence as shown in SEQ ID NO: 6 by substitution, deletion, insertion, or addition of one or several amino acids and encoding a protein having alcohol dehydrogenase activity. The term “several” used herein refers to, for example, 2 to 30, preferably 2 to 20, more preferably 2 to 10, and most preferably 2 to 5.

Furthermore, the alcohol dehydrogenase genes are not limited to the genes identified by SEQ ID NOs: 5 and 6. For example, it may be a gene hybridizing under stringent conditions to the full-length sequence or a partial sequence of a complementary strand of DNA comprising the nucleotide sequence as shown in SEQ ID NO: 5 and encoding a protein having alcohol dehydrogenase activity. Under “stringent conditions,” so-called specific hybrids are formed, but non-specific hybrids are not formed. Such conditions can be adequately determined with reference to, for example, Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42° C. to 68° C. and preferably 42° C. to 65° C., for example. Further specifically, the sodium concentration is 5×SSC (83 mM NaCl, 83 mM sodium citrate), and the temperature is 42° C.

As described above, whether or not a gene comprising a nucleotide sequence that differs from the sequence shown in SEQ ID NO: 5 or a gene encoding an amino acid sequence that differs from the sequence shown in SEQ ID NO: 6 would function as an alcohol dehydrogenase gene having activity of converting acetaldehyde into ethanol may be determined by, for example, preparing an expression vector comprising the gene of interest incorporated into an adequate site between a promoter and a terminator, transforming a yeast host using such expression vector, and assaying alcohol dehydrogenase activity of the protein expressed. Alcohol dehydrogenase activity of converting acetaldehyde into ethanol can be assayed by preparing a solution containing aldehyde and NADH or NADPH as substrates, allowing the target protein to react at adequate temperature, and assaying the generated alcohol or spectroscopically assaying NAD⁺ or NADP⁺.

A recombinant yeast strain used in the method for producing ethanol of the present invention is preferably characterized by a lowered expression level of the alcohol dehydrogenase gene (the ADH2 gene) having activity of converting ethanol into aldehyde. In order to lower the expression level of such gene, a promoter of the inherent gene of interest may be modified, or such gene may be deleted. In order to delete the gene, either or both of a pair of ADH2 genes present in diploid recombinant yeast may be deleted. Examples of techniques for suppressing gene expression include the transposon technique, the transgene technique, post-transcriptional gene silencing, the RNAi technique, the nonsense mediated decay (NMD) technique, the ribozyme technique, the anti-sense technique, the miRNA (micro-RNA) technique, and the siRNA (small interfering RNA) technique.

The nucleotide sequence of the ADH2 gene of Saccharomyces cerevisiae and the amino acid sequence of a protein encoded by such gene are shown in SEQ ID NOs: 7 and 8, respectively. The target alcohol dehydrogenase genes are not limited to the genes identified by SEQ ID NOs: 7 and 8. It may be a paralogous gene or a homologous gene in the narrow sense having different nucleotide and amino acid sequences.

The alcohol dehydrogenase genes are not limited to the genes identified by SEQ ID NOs: 7 and 8. For example, it may be a gene comprising an amino acid sequence having 70% or higher, preferably 80% or higher, more preferably 90% or higher, and most preferably 95% or higher sequence similarity to or identity with the amino acid sequence as shown in SEQ ID NO: 8 and encoding a protein having alcohol dehydrogenase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by such amino acid residues.

Further, the alcohol dehydrogenase genes are not limited to the genes identified by SEQ ID NOs: 7 and 8. For example, it may be a gene comprising an amino acid sequence derived from the amino acid sequence as shown in SEQ ID NO: 8 by substitution, deletion, insertion, or addition of one or several amino acids and encoding a protein having alcohol dehydrogenase activity. The term “several” used herein refers to, for example, 2 to 30, preferably 2 to 20, more preferably 2 to 10, and most preferably 2 to 5.

Furthermore, the alcohol dehydrogenase genes are not limited to the genes identified by SEQ ID NOs: 7 and 8. For example, it may be a gene hybridizing under stringent conditions to the full-length sequence or a partial sequence of a complementary strand of DNA comprising the nucleotide sequence as shown in SEQ ID NO: 7 and encoding a protein having alcohol dehydrogenase activity. Under “stringent conditions,” so-called specific hybrids are formed, but non-specific hybrids are not formed. Such conditions can be adequately determined with reference to, for example, Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42° C. to 68° C. and preferably 42° C. to 65° C., for example. Further specifically, the sodium concentration is 5×SSC (83 mM NaCl, 83 mM sodium citrate), and the temperature is 42° C.

As described above, whether or not a gene comprising a nucleotide sequence that differs from the sequence shown in SEQ ID NO: 7 or a gene encoding an amino acid sequence that differs from the sequence shown in SEQ ID NO: 8 would function as an alcohol dehydrogenase gene having activity of converting ethanol into aldehyde may be determined by, for example, preparing an expression vector comprising the gene of interest incorporated into an adequate site between a promoter and a terminator, transforming a yeast host using such expression vector, and assaying alcohol dehydrogenase activity of the protein expressed. Alcohol dehydrogenase activity of converting ethanol into aldehyde can be assayed by preparing a solution containing alcohol and NAD+ or NADP+ as substrates, allowing the target protein to react at adequate temperature, and assaying the generated aldehyde or spectroscopically assaying NADH or NADPH.

Further examples of other genes that can be introduced into a recombinant yeast strain include genes associated with the metabolic pathway of L-arabinose, which is a pentose contained in hemicellulose constituting a biomass. Examples of such genes include an L-arabinose isomerase gene, an L-ribulokinase gene, and an L-ribulose-5-phosphate-4-epimerase gene derived from prokaryotes and an L-arabitol-4-dehydrogenase gene and an L-xylose reductase gene derived from eukaryotes.

In particular, an example of another gene to be introduced into a recombinant yeast strain is a gene capable of promoting the use of xylose in a medium. A specific example thereof is a gene encoding xylulokinase having activity of generating xylulose-5-phosphate using xylulose as a substrate. The metabolic flux of the pentose phosphate pathway can be improved through the introduction of the xylulokinase gene.

Further, a gene encoding an enzyme selected from the group of enzymes constituting a non-oxidative process in the pentose phosphate pathway can be introduced into a recombinant yeast strain. Examples of enzymes constituting a non-oxidative process in the pentose phosphate pathway include ribose-5-phosphate isomerase, ribulose-5-phosphate-3-epimerase, transketolase, and transaldolase. It is preferable that one or more genes encoding such enzymes be introduced. It is more preferable to introduce two or more such genes in combination, further preferable to introduce three or more genes in combination, and the most preferable to introduce all of the genes above.

More specifically, the xylulokinase (XK) gene of any origin can be used without particular limitation. A wide variety of microorganisms, such as bacterial and yeast strains, which assimilate xylulose, possess the XK gene. Preferable examples of such genes include the XK genes derived from yeast strains, lactic acid bacteria, E. coli bacteria, and plants. Information concerning XK genes can be obtained by searching the website of NCBI or other institutions, according to need. An example of an XK gene is XKS1, which is an XK gene derived from the S. cerevisiae S288C strain (GenBank: Z72979) (the nucleotide sequence and the amino acid sequence in the CDS coding region).

More specifically, a transaldolase (TAL) gene, a transketolase (TKL) gene, a ribulose-5-phosphate epimerase (RPE) gene, and a ribose-5-phosphate ketoisomerase (RKI) gene of any origin can be used without particular limitation. A wide variety of organisms comprising the pentose phosphate pathway possess such genes. For example, a common yeast strain such as S. cerevisiae possesses such genes. Information concerning such genes can be obtained from the website of NCBI or other institutions, according to need. Genes belonging to the same genus as the host eukaryotic cells, such as eukaryotic or yeast cells, are preferable, and genes originating from the same species as the host eukaryotic cells are further preferable. A TAL1 gene, a TKL1 gene and a TKL2 gene, an RPE1 gene, and an RKI gene can be preferably used as the TAL gene, the TKL genes, the RPE gene, and the RKI gene, respectively. Examples of such genes include a TAL1 gene derived from the S. cerevisiae S288 strain (GenBank: U19102), a TKL1 gene derived from the S. cerevisiae S288 strain (GenBank: X73224), an RPE1 gene derived from the S. cerevisiae S288 strain (GenBank: X83571), and an RKI1 gene derived from the S. cerevisiae S288 strain (GenBank: Z75003).

### <Production of Recombinant Yeast Strain>

The xylose isomerase gene and the acetaldehyde dehydrogenase gene are introduced into a host yeast genome, and a recombinant yeast strain that can be used in the present invention can be produced. The xylose isomerase gene and the acetaldehyde dehydrogenase gene may be introduced into a yeast strain that does not have xylose-metabolizing ability, a yeast strain that inherently has xylose-metabolizing ability, or a yeast strain that does not have xylose-metabolizing ability together with the xylose metabolism-associated gene. When the xylose isomerase gene, the acetaldehyde dehydrogenase gene, and the genes described above are introduced into a yeast strain, such genes may be simultaneously introduced thereinto, or such genes may be successively introduced with the use of different expression vectors.

Examples of host yeast strains that can be used include, but are not particularly limited to, Candida Shehatae, Pichia stipitis, Pachysolen tannophilus, Saccharomyces cerevisiae, and Schizosaccaromyces pombe, with Saccharomyces cerevisiae being particularly preferable. Experimental yeast strains may also be used from the viewpoint of experimental convenience, or industrial (practical) strains may also be used from the viewpoint of practical usefulness. Examples of industrial strains include yeast strains used for the production of wine, sake, and shochu.

Use of a host yeast strain having homothallic properties is preferable. According to the technique disclosed in JP 2009-34036 A, multiple copies of genes can be easily introduced into a genome with the use of a yeast strain having homothallic properties. The term “yeast strain having homothallic properties” has the same meaning as the term “homothallic yeast strain.” Yeast strains having homothallic properties are not particularly limited, and any yeast strains can be used. An example of a yeast strain having homothallic properties is the Saccharomyces cerevisiae OC-2 train (NBRC2260), but yeast strains are not limited thereto. Examples of other yeast strains having homothallic properties include an alcohol-producing yeast (Taiken No. 396, NBRC0216) (reference: “Alcohol kobo no shotokusei” (“Various properties of alcohol-producing yeast”), Shuken Kaiho, No. 37, pp. 18-22, 1998.8), an ethanol-producing yeast isolated in Brazil and in Japan (reference: “Brazil to Okinawa de bunri shita Saccharomyces cerevisiae yaseikabu no idengakuteki seishitsu” (“Genetic properties of wild-type Saccharomyces cerevisiae isolated in Brazil and in Okinawa”), the Journal of the Japan Society for Bioscience, Biotechnology, and Agrochemistry, Vol. 65, No. 4, pp. 759-762, 1991.4), and 180 (reference: “Alcohol Hakkoryoku no tsuyoi kobo no screening” (“Screening of yeast having potent alcohol-fermenting ability”), the Journal of the Brewing Society of Japan, Vol. 82, No. 6, pp. 439-443, 1987.6). In addition, the HO gene may be introduced into a yeast strain exhibiting heterothallic phenotypes in an expressible manner, and the resulting strain can be used as a yeast strain having homothallic properties. That is, the term “yeast strain having homothallic properties” used herein also refers to a yeast strain into which the HO gene has been introduced in an expressible manner.

The Saccharomyces cerevisiae OC-2 strain is particularly preferable since it has heretofore been used for wine brewing, and the safety thereof has been verified. As described in the examples below, the Saccharomyces cerevisiae OC-2 strain is preferable in terms of its excellent promoter activity at high sugar concentrations. In particular, the Saccharomyces cerevisiae OC-2 strain is preferable in terms of its excellent promoter activity for the pyruvate decarboxylase gene (PDC1) at high sugar concentrations.

Promoters of genes to be introduced are not particularly limited. For example, promoters of the glyceraldehyde-3-phosphate dehydrogenase gene (TDH3), the 3-phosphoglycerate kinase gene (PGK1), and the high-osmotic pressure response 7 gene (HOR7) can be used. The promoter of the pyruvate decarboxylase gene (PDC1) is particularly preferable in terms of its high capacity for expressing target genes in a downstream region at high levels.

Specifically, such gene may be introduced into the yeast genome together with an expression-regulating promoter or another expression-regulated region. Such gene may be introduced into a host yeast genome in such a manner that expression thereof is regulated by a promoter or another expression-regulated region of a gene that is inherently present therein.

The gene can be introduced into the genome by any conventional technique known as a yeast transformation technique. Specific examples include, but are not limited to, electroporation (Meth. Enzym., 194, p. 182, 1990), the spheroplast technique (Proc. Natl. Acad. Sci., U.S.A., 75, p. 1929, 1978), and the lithium acetate method (J. Bacteriology, 153, p. 163, 1983; Proc. Natl. Acad. Sci., U.S.A., 75, p. 1929, 1978; Methods in yeast genetics, 2000 Edition: A Cold Spring Harbor Laboratory Course Manual).

### <Production of Ethanol>

When producing ethanol with the use of the recombinant yeast strain described above, ethanol fermentation is carried out by culture in a medium containing at least xylose. A medium in which ethanol fermentation is carried out contains at least xylose as a carbon source. The medium may contain another carbon source, such as glucose in advance.

Xylose that is contained in a medium to be used for ethanol fermentation can be derived from a biomass. In other words, a medium to be used for ethanol fermentation may comprise a cellulosic biomass and hemicellulase that generates xylose through saccharification of hemicellulose contained in a cellulosic biomass. The cellulosic biomass may have been subjected to a conventional pretreatment technique. Examples of pretreatment techniques include, but are not particularly limited to, degradation of a lignin with a microorganism and grinding of a cellulosic biomass. For example, a ground cellulosic biomass may be subjected to pretreatment, such as soaking thereof in a dilute sulfuric acid solution, alkaline solution, or ionic solution, hydrothermal treatment, or fine grinding. Thus, the efficiency of biomass saccharification can be improved.

When producing ethanol with the use of the recombinant yeast strain described above, the medium may further comprise cellulose and cellulase. In such a case, the medium would contain glucose generated by the action of cellulase imposed upon cellulose. When a medium used for ethanol fermentation contains cellulose, such cellulose can be derived from a biomass. In other words, a medium used for ethanol fermentation may comprise cellulase that is capable of saccharifying cellulase contained in a cellulosic biomass.

A saccharified solution resulting from saccharification of a cellulosic biomass may be added to the medium used for ethanol fermentation. such a case, the saccharified solution contains remaining cellulose or cellulase and xylose derived from hemicellulose contained in a cellulosic biomass.

As described above, the method for producing ethanol of the present invention comprises a step of ethanol fermentation involving the use of at least xylose as a saccharide source. According to the method for producing ethanol of the present invention, ethanol can be produced through ethanol fermentation using xylose as a saccharide source. According to the method for producing ethanol with the use of the recombinant yeast strain of the present invention, ethanol fermentation is followed by recovery of ethanol from the medium. Ethanol may be recovered by any conventional means without particular limitation. After the completion of the process of ethanol fermentation mentioned above, for example, a liquid layer containing ethanol is separated from a solid layer containing the recombinant yeast strain or solid matter via solid-solution separation. Thereafter, ethanol contained in a liquid layer is separated and purified by distillation, so that highly purified ethanol can be recovered. The degree of ethanol purification can be adequately determined in accordance with the purpose of use of the ethanol.

When producing ethanol with the use of a saccharide derived from a biomass, in general, a fermentation inhibitor, such as acetic acid or furfural, may occasionally be generated in the process of pretreatment or saccharification. In particular, acetic acid is known to inhibit the growth and multiplication of yeast strains and to lower the efficiency for ethanol fermentation conducted with the use of xylose as a saccharide source.

According to the present invention, however, recombinant yeast strains into which the xylose isomerase gene and the acetaldehyde dehydrogenase gene have been introduced are used. Thus, acetic acid contained in a medium can be metabolized, and acetic acid concentration in a medium can be maintained at a low level. Accordingly, the method for producing ethanol of the present invention can achieve an ethanol yield superior to that achieved with the use of yeast strains into which neither a xylose isomerase gene nor an acetaldehyde dehydrogenase gene have been introduced.

According to the method for producing ethanol of the present invention, acetic acid concentration in a medium remains low after the recombinant yeast strain has been cultured for a given period of time. Even if part of the medium after such given period of time is used for a continuous culture system in which a new culture process is initiated, accordingly, the amount of acetic acid carry-over can be reduced. According to the method for producing ethanol of the present invention, therefore, the amount of acetic acid carry-over can be reduced even when cells are recovered and reused after the completion of the process of ethanol fermentation.

The method for producing ethanol of the present invention may employ the so-called simultaneous saccharification and fermentation process, in which the step of saccharification of cellulose contained in a medium with a cellulase proceeds concurrently with the process of ethanol fermentation carried out with the use of saccharide sources (i.e., xylose and glucose generated by saccharification). With the simultaneous saccharification and fermentation process, the step of saccharification of a cellulosic biomass is carried out simultaneously with the process of ethanol fermentation.

Methods of saccharification are not particularly limited, and, for example, an enzymatic method involving the use of a cellulase preparation, such as cellulase or hemicellulase, may be employed. A cellulase preparation contains a plurality of enzymes involved in degradation of a cellulose chain and a hemicellulose chain, and it exhibits a plurality of types of activity, such as endoglucanase activity, endoxylanase activity, cellobiohydrolase activity, glucosidase activity, and xylosidase activity. Cellulase preparations are not particularly limited, and examples include cellulases produced by Trichoderma reesei and Acremonium cellulolyticus. Commercially available cellulase preparations may also be used.

In the simultaneous saccharification and fermentation process, a cellulase preparation and the recombinant microorganism are added to a medium containing a cellulosic biomass (a biomass after pretreatment may be used), and the recombinant yeast strain is cultured at a given temperature. Culture may be carried out at any temperature without particular limitation, and the temperature may be 25° C. to 45° C., and preferably 30° C. to 40° C. from the viewpoint of ethanol fermentation efficiency. The pH level of the culture solution is preferably 4 to 6. When conducting culture, stirring or shaking may be carried out. Alternatively, the simultaneous saccharification and fermentation process may be carried out irregularly in such a manner that saccharification is first carried out at an optimal temperature for an enzyme (40° C. to 70° C.), temperature is lowered to a given level (30° C. to 40° C.), and a yeast strain is then added thereto.

### EXAMPLES

Hereafter, the present invention is described in greater detail with reference to the examples, although the technical scope of the present invention is not limited to these examples.

### Example 1

In the present example, a recombinant yeast strain was prepared through introduction of a xylose isomerase gene and an acetaldehyde dehydrogenase gene of E. coli (the mhpF gene), and the acetic acid metabolizing ability of the recombinant yeast strain was evaluated.

### <Production of Vectors for Gene Introduction>

### (1) Vector for XKS1 Gene Introduction

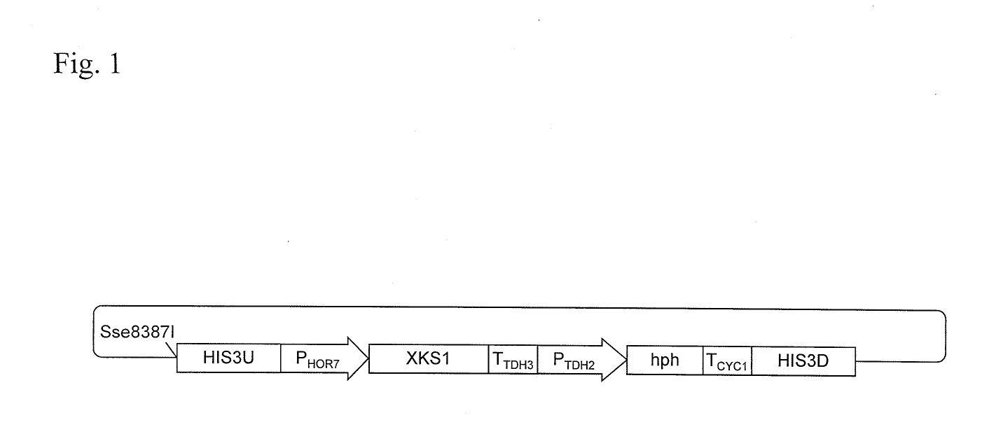

As a vector for introducing the xylulokinase (XK) gene derived from S. cerevisiae into a yeast strain, the pUC-HIS3D-P_HOR7-XKS1-T_TDH3-P_TDH2-hph-T_CYC1-HIS3D vector shown in FIG. 1 was produced. This vector comprises: the XKS1 gene, which is a XK gene derived from the S. cerevisiae NBRC304 strain in which the HOR7 promoter and the TDH3 terminator are added on the 5′ side and the 3′ side, respectively (GenBank: X61377); an upstream region of approximately 500 by (HIS3D) of the histidine synthetase (HIS3) gene and a region of approximately 500 by within such gene (HIS3D), which are regions to be integrated into the yeast genome via homologous recombination; and the hygromycin phosphotransferase (hph) gene (a marker gene) in which the TDH2 promoter and the CYC1 terminator are added on the 5′ side and the 3′ side, respectively. The Sse8387I restriction enzyme sites were introduced into sites outside the homologous recombination region. The nucleotide sequence of the coding region of the XKS1 gene derived from the S. cerevisiae NBRC304 strain and the amino acid sequence of xylulokinase encoded by such gene are shown in SEQ ID NOs: 9 and 10, respectively.

### (2) Vector for XI Gene Introduction

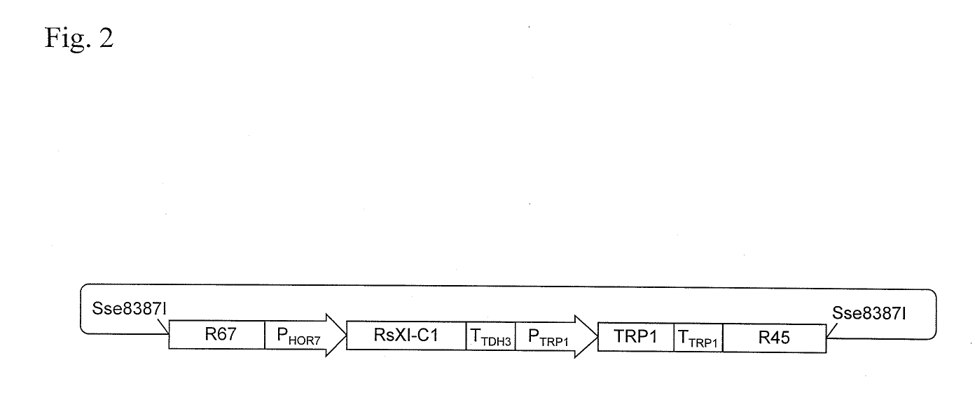

As a vector for introducing the xylose isomerase gene derived from the intestinal protozoa of Reticulitermes speratus (RsXI-C1; see JP 2011-147445 A), the pUC-R67-HOR7p-RsXI-T_TDH3-TRP1d-R45 vector shown in FIG. 2 was produced. This vector comprises: the RsXI-C1 gene in which the HOR7 promoter and the TDH3 terminator are added on the 5′ side and the 3′ side, respectively; R45 and R67 of homologous sequences to the rRNA gene (rDNA), which are regions to be integrated into the yeast genome via homologous recombination; and the TRP1d marker gene exhibiting a lowered expression level as a result of disruption of the promoter region. The Sse8387I restriction enzyme sites were introduced into sites outside the homologous recombination region. Multiple copies of genes including RsXI-C1 are introduced into the rDNA locus of the chromosome 12 with the aid of R45 and R67. The TRP1d marker can function as a marker if multiple copies thereof are introduced into the chromosome. With the use of such vector, accordingly, multiple copies of genes can be introduced. The RsXI-C1 gene used in this example was prepared by the total synthesis on the basis of the nucleotide sequence designed by changing codons over the entire region in accordance with the frequency of codon usage of the yeast strain. The nucleotide sequence of the RsXI-C1 gene designed in the present example and the amino acid sequence of xylose isomerase encoded by such gene are shown in SEQ ID NOs: 3 and 4, respectively.

### (3) Vector for TAL1 and TKL1 Gene Introduction

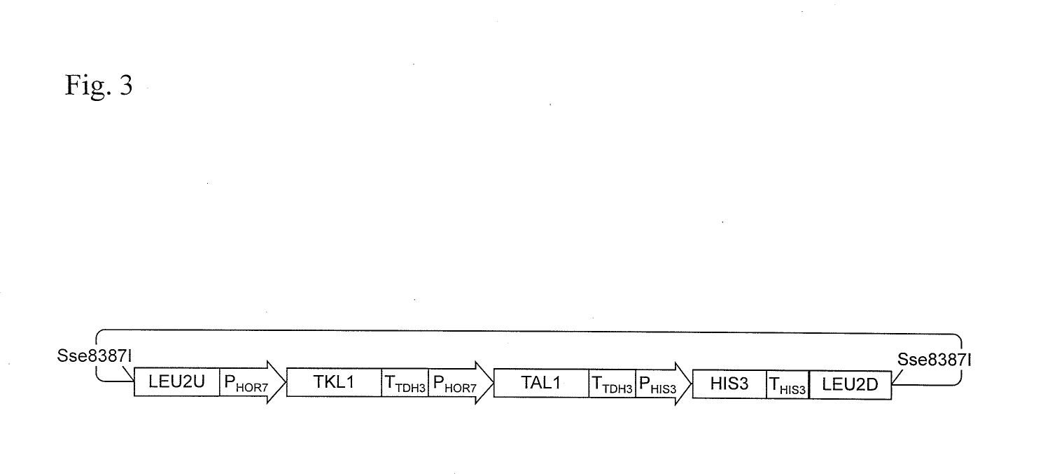

As a vector for introducing the transaldolase 1 (TAL1) gene and the transketolase 1 (TKL1) gene derived from S. cerevisiae into a yeast strain, the pUC-LEU2U-P_HOR7-TAL1-T_TDH3-P_HOR7-TKL1-T_TDH3-HIS3-LEU2 D vector shown in FIG. 3 was produced. This vector comprises: the TAL1 gene derived from the S. cerevisiae S288 strain in which the HOR7 promoter and the TDH3 terminator are added on the 5′ side and the 3′ side, respectively (GenBank: U19102); the TKL1 gene derived from the S. cerevisiae S288 strain in which the HOR7 promoter and the TDH3 terminator are added on the 5′ side and the 3′ side, respectively (GenBank: X73224); an upstream region of approximately 500 by from the 3′ terminus of the leucine synthetase (LEU2) gene and an upstream region of approximately 450 by from the 5′ terminus thereof (LEU2D), which are regions to be integrated into the yeast genome via homologous recombination; and the histidine synthetase (HIS3) gene (a marker gene). The Sse8387I restriction enzyme sites were introduced into sites outside the homologous recombination region. The nucleotide sequence of the coding region of the TAL1 gene derived from the S. cerevisiae S288 strain and the amino acid sequence of transaldolase 1 encoded by such gene are shown in SEQ ID Nos: 11 and 12, respectively. The nucleotide sequence of the coding region of the TKL1 gene derived from the S. cerevisiae S288 strain and the amino acid sequence of transketolase 1 encoded by such gene are shown in SEQ ID Nos: 13 and 14, respectively.

(4) Vector for RPE1 and RKI1 gene introduction and GRE3 gene disruption

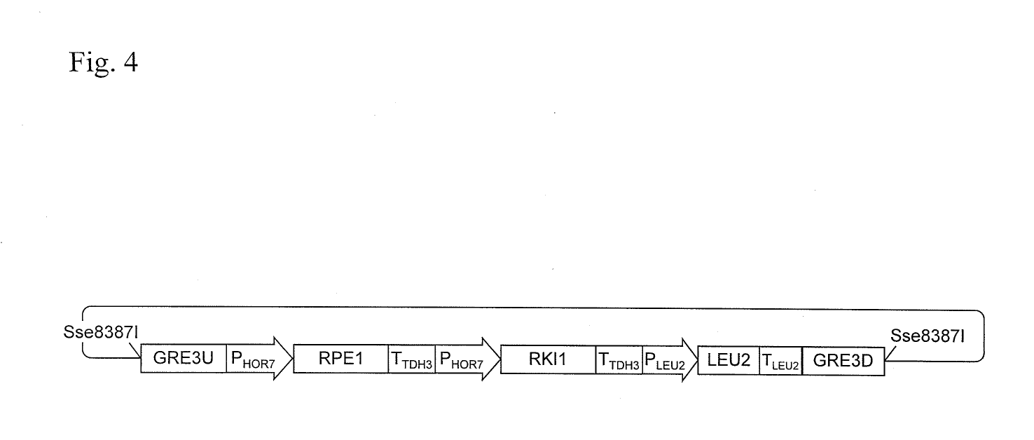

As a vector for introducing the ribulose phosphate epimerase 1 (RPE1) gene and the ribose phosphate ketoisomerase (RKI1) gene derived from S. cerevisiae into a yeast strain, the pUC-GRE3U-P_HOR7-RPE1-T_TDH3-P_HOR7-RKI1-T_TDH3-LEU2-GRE3 D vector shown in FIG. 4 was produced. This vector comprises: the RPE1 gene derived from the S. cerevisiae S288 strain in which the HOR7 promoter and the TDH3 terminator are added on the 5′ side and the 3′ side, respectively (GenBank: X83571); the RKI1 gene derived from the S. cerevisiae S288 strain in which the HOR7 promoter and the TDH3 terminator are added on the 5′ side and the 3′ side, respectively (GenBank: Z75003); a region of approximately 800 by comprising the 3′ terminal region of approximately 500 by of the GRE3 gene and an upstream region of approximately 1,000 by of the GRE3 gene (GRE3D), which are regions to be integrated into the yeast genome via homologous recombination and for disruption of the aldose reductase 3 (GRE3) gene; and the leucine synthetase (LEU2) gene (a marker gene). The Sse8387I restriction enzyme sites were introduced into sites outside the homologous recombination region. The nucleotide sequence of the coding region of the RPE1 gene derived from the S. cerevisiae S288 strain and the amino acid sequence of ribulose phosphate epimerase 1 encoded by such gene are shown in SEQ ID Nos: 15 and 16, respectively. Further, the nucleotide sequence of the coding region of the RKI1 gene derived from the S. cerevisiae S288 strain and the amino acid sequence of ribose phosphate ketoisomerase encoded by such gene are shown in SEQ ID Nos: 17 and 18, respectively.

### (5) Vector for ADH2 Gene Disruption

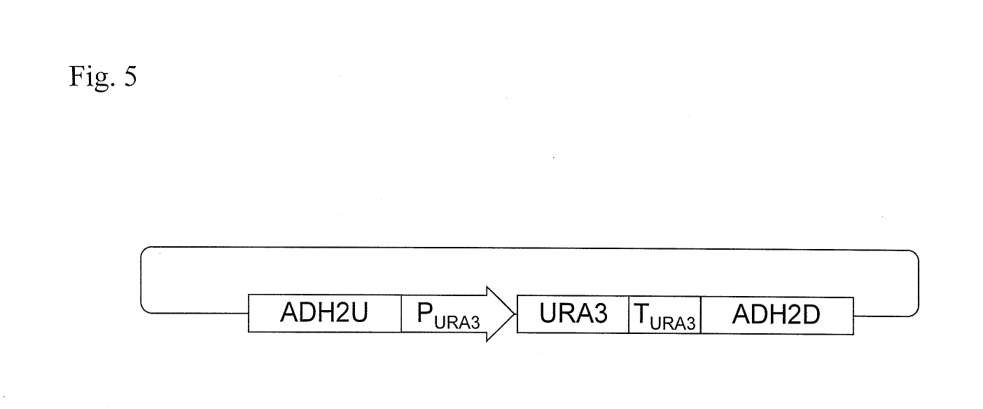

As a vector for disrupting the ADH2 gene inherent in the host, the pCR-ADH2U-URA3-ADH2D vector shown in FIG. 5 was produced. This vector comprises regions to be integrated into the yeast genome via homologous recombination and for disruption of the alcohol dehydrogenase 2 (ADH2) gene: i.e., an upstream region of approximately 700 by of the ADH2 gene (ADH2U); a downstream region of approximately 800 by of the ADH2 gene (ADH2D); and the orotidine-5′-phosphate decarboxylase (URA3) gene (a marker gene).

### (6) Vector for ADH1 Gene Introduction

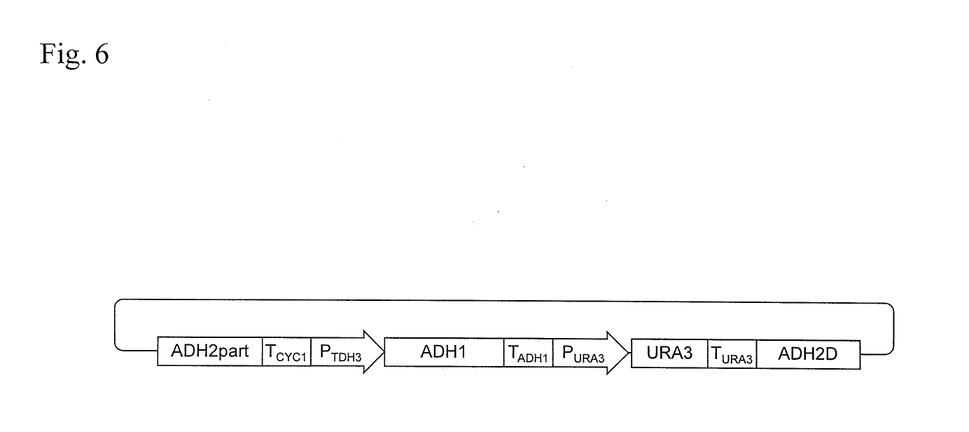

As a vector for introducing the alcohol dehydrogenase 1 (ADH1) gene into a yeast strain, the pCR-ADH2part-T_CYC1-P_TDH3-ADH1-T_ADH1-URA3-ADH2D vector shown in FIG. 6 was produced. This vector comprises: the ADH1 gene derived from the S. cerevisiae S288 strain in which the TDH3 promoter and the ADH1 terminator are added on the 5′ side and the 3′ side, respectively (GenBank: Z74828.1); an upstream region of approximately 450 by from the 3′ terminus (ADH2part) and a downstream region of approximately 700 by from the 3′ terminus (ADH2D) of the ADH2 gene, which are regions to be integrated into the yeast genome via homologous recombination; the CYC1 terminator as the ADH2 terminator; and the URA3 gene (a marker gene).

(7) Vector for mhpF Gene Introduction

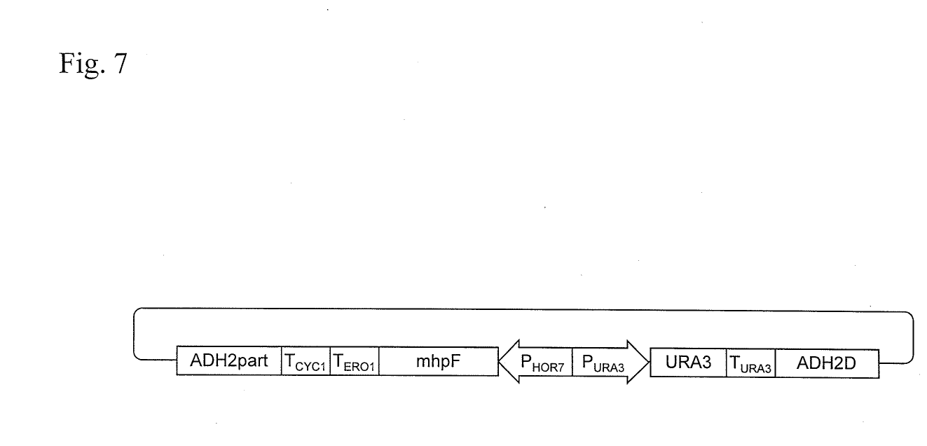

As a vector for introducing the acetaldehyde dehydrogenase (mhpF) gene derived from E. coli into a yeast strain, the pCR-ADH2part-T_CYC1-ERO1_T-mhpF-HOR7_P-URA3-ADH2D vector shown in FIG. 7 was produced. This vector comprises: the acetaldehyde dehydrogenase gene derived from E. coli in which the HOR7 promoter and the ERO1 terminator are added on the 5′ side and the 3′ side, respectively (the mhpF gene); an upstream region of approximately 450 by from the 3′ terminus (ADH2part) and a downstream region of approximately 700 by from the 3′ terminus (ADH2D) of the ADH2 gene, which are regions to be integrated into the yeast genome via homologous recombination; the CYC1 terminator as the ADH2 terminator; and the URA3 gene (a marker gene). The mhpF gene used in this example was prepared by the total synthesis on the basis of the nucleotide sequence designed by changing codons over the entire region in accordance with the frequency of codon usage of the yeast strain. The nucleotide sequence of the mhpF gene designed in the present example and the amino acid sequence of acetaldehyde dehydrogenase encoded by such gene are shown in SEQ ID NOs: 1 and 2, respectively.

(8) Vector for mhpF and ADH1 gene introduction

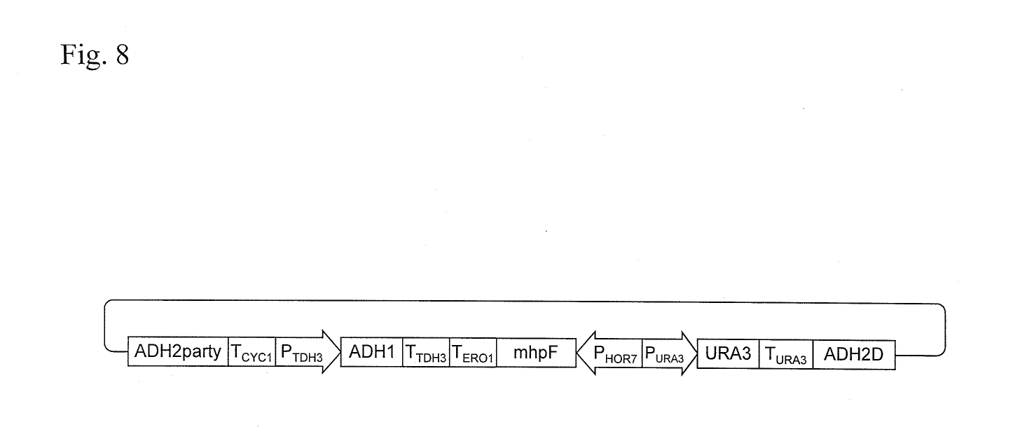

As a vector for introducing the mhpF gene and the ADH1 gene into a yeast strain, the pCR-ADH2part-T_CYC1-P_TDH3-ADH1-T_ADH1-ERO1_T-mhpF-HOR7_P-URA3-ADH2D vector shown in FIG. 8 was produced. This vector comprises: the mhpF gene in which the HOR7 promoter and the ERO1 terminator are added on the 5′ side and the 3′ side, respectively (same as (7) above); the ADH1 gene derived from S. cerevisiae S288 strain in which the TDH3 promoter and the ADH1 terminator are added on the 5′ side and the 3′ side, respectively (same as (6) above); an upstream region of approximately 450 by from the 3′ terminus (ADH2part) and a downstream region of approximately 700 by from the 3′ terminus (ADH2D) of the ADH2 gene, which are regions to be integrated into the yeast genome via homologous recombination; the CYC1 terminator as the ADH2 terminator; and the URA3 gene (a marker gene).

(9) Vector for mhpF Gene Introduction and ADH2 Gene Disruption

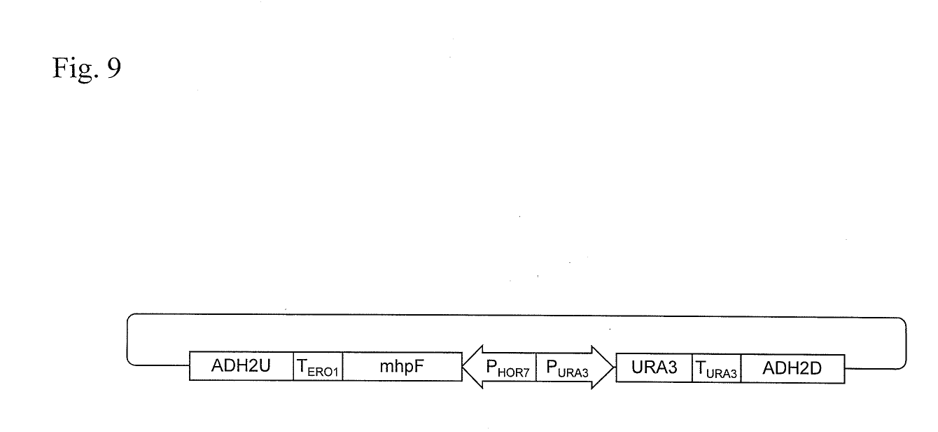

As a vector for introducing the mhpF gene into a yeast strain and for disrupting the ADH2 gene, the pCR-ADH2D-ERO1_T-mhpF-HOR7_P-URA3-ADH2D vector shown in FIG. 9 was produced. This vector comprises: the mhpF gene in which the HOR7 promoter and the ERO1 terminator are added on the 5′ side and the 3′ side, respectively (same as (7) above); an upstream region of approximately 700 by (ADH2D) and an upstream region of approximately 800 by (ADH2D) of the ADH2 gene, which are regions to be integrated into the yeast genome via homologous recombination and for disruption of the ADH2 gene; and the URA3 gene (a marker gene).

(10) Vector for mhpF and ADH1 Gene Introduction and ADH2 Gene Disruption

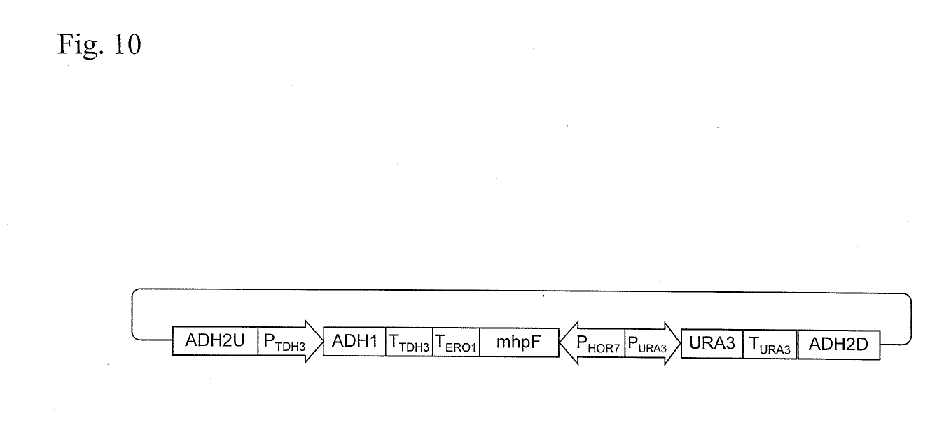

As a vector for introducing the mhpF and ADH1 genes into a yeast strain and for disrupting the ADH2 gene, the pCR-ADH2D-P_TDH3-ADH1-T_ADH1-ERO1_T-mhpF-HOR7_P-URA3-ADH 2D vector shown in FIG. 10 was produced. This vector comprises: the mhpF gene in which the HOR7 promoter and the ERO1 terminator are added on the 5′ side and the 3′ side, respectively (same as (7) above); the ADH1 gene derived from S. cerevisiae S288 strain in which the TDH3 promoter and the ADH1 terminator are added on the 5′ side and the 3′ side, respectively (same as (6) above); an upstream region of approximately 700 by (ADH2D) and an upstream region of approximately 800 by (ADH2D) of the ADH2 gene, which are regions to be integrated into the yeast genome via homologous recombination and for disruption of the ADH2 gene; and the URA3 gene (a marker gene).

### (11) Control Vector (Marker Gene Only)

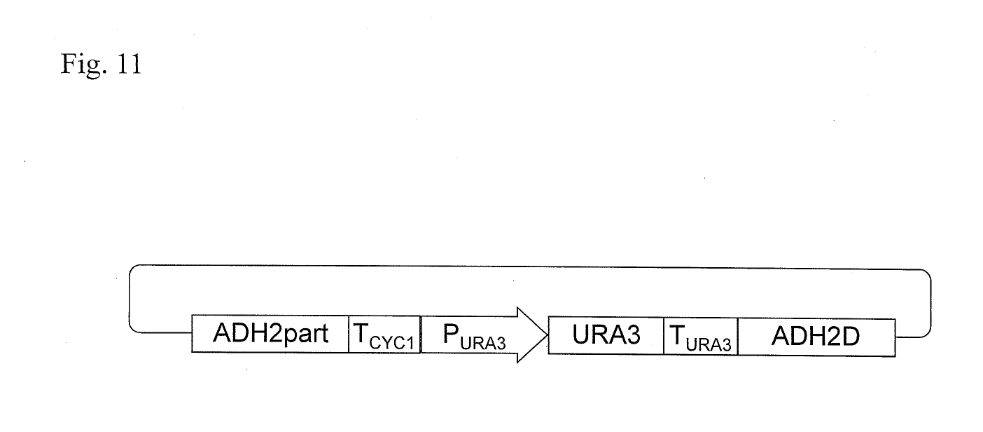

As a control vector intended to selectively introduce a marker gene, the pCR-ADH2part-T_CYC1-URA3-ADH2D vector shown in FIG. 11 was produced. This vector comprises: an upstream region of approximately 450 by from the 3′ terminus (ADH2part) and a downstream region of approximately 700 by from the 3′ terminus (ADH2D) of the ADH2 gene, which are regions to be integrated into the yeast genome via homologous recombination; the CYC1 terminator as the ADH2 terminator; and the URA3 gene (a marker gene).

### <Production of Yeast Strains Comprising Vectors Introduced Thereinto>

The diploid yeast strains, Saccharomyces cerevisiae OC2-T (Saitoh, S. et al., J. Ferment. Bioeng., 1996, vol. 81, pp. 98-103), were selected in a 5-fluoroorotic acid-supplemented medium (Boeke, J. D., et al., 1987, Methods Enzymol., 154: 164-75.), and uracil auxotrophic strains were designated as host strains.

Yeast strains were transformed using the Frozen-EZ Yeast Transformation II (ZYMO RESEARCH) in accordance with the protocols included thereinto. At the outset, the pUC-HIS3U-P_HOR7-XKS1-T_TDH3-P_TDH2-hph-T_CYC1-HIS3D vector was digested with the Sse8387I restriction enzyme, the OC2-T strains were transformed using the resulting digestion fragment, the resulting transformants were applied to a YPD+HYG agar medium, and the grown colonies were then subjected to acclimatization. The acclimatized elite strains were designated as the OC100 strains. Subsequently, the pUC-LEU2U-P_HOR7-TAL1-T_TDH3-P_HOR7-TKL1-T_TDH3-HIS3-LEU2 D vector was digested with the Sse8387I restriction enzyme, the OC100 strains were transformed using the resulting digestion fragment, the resulting transformants were applied to a histidine-free SD agar medium (Methods in Yeast Genetics, Cold Spring Harbor Laboratory Press), and the grown colonies were then subjected to acclimatization. The acclimatized elite strains were designated as the OC300 strains. Subsequently, the pUC-GRE3U-P_HOR7-RPE1-T_TDH3-P_HOR7-RKI1-T_TDH3-LEU2-GRE3 D vector was digested with the Sse8387I restriction enzyme, the OC300 strains were transformed using the resulting digestion fragment, the resulting transformants were applied to a leucine-free SD agar medium, and the grown colonies were then subjected to acclimatization. The acclimatized elite strains were designated as the OC600 strains. Subsequently, the pUC-R67-HOR7p-RsXI-T_TDH3-TRP1d-R45 vector was digested with the Sse8387I restriction enzyme, the OC600 strains were transformed using the resulting digestion fragment, the resulting transformants were applied to a tryptophan-free SD agar medium, and the grown colonies were then subjected to acclimatization. The acclimatized elite strains were designated as the OC700 strains. The thus-produced OC700 strains comprise the RsXI-C1 gene, the XK gene, the TAL1 gene, the TKL1 gene, the RPE1 gene, and the RKI1 gene introduced thereinto.

Subsequently, regions between homologous recombination sites of the vectors pCR-ADH2U-URA3-ADH2D, pCR-ADH2part-T_CYC1-P_TDH3-ADH1-T_ADH1-URA3-ADH2D, pCR-ADH2part-T_CYC1-ERO1_T-mhpF-HOR7_P-URA3-ADH2D, pCR-ADH2part-T_CYC1-P_TDH3-ADH1-T_ADH1-ERO1_T-mhpF-HOR7_P-URA3-ADH2D, pCR-ADH2U-ERO1_T-mhpF-HOR7_P-URA3-ADH2D, pCR-ADH2U-P_TDH3-ADH1-T_ADH1-ERO1_T-mhpF-HOR7_P-URA3-ADH 2D, and pCR-ADH2part-T_CYC1-URA3-ADH2D were amplified by PCR, the resulting amplified fragments were used to transform the OC700 strains, the resulting transformants were applied to a uracil-free SD agar medium, and the grown colonies were then subjected to acclimatization. The acclimatized elite strains were designated as the Uz1048 strains, the Uz1047 strains, the Uz928 strains, the Uz1012 strains, the Uz926 strains, the Uz736 strains, and the Uz1049 strains, respectively.

### <Fermentation Test>

From among the Uz1048, Uz1047, Uz928, Uz1012, Uz926, Uz736, and Uz1049 strains obtained in the manner described above, strains exhibiting high fermentation ability were selected and subjected to a fermentation test in flasks in the manner described below. The test strains were inoculated into 100-ml baffled flasks each comprising 20 ml of YPD liquid medium (glucose concentration: 20 g/l; yeast extract concentration: 10 g/l; and peptone concentration: 20 g/l), and culture was conducted at 30° C. and 120 rpm for 24 hours. The strains were harvested and inoculated into 20-ml flasks each comprising 10 ml of D20X6OYAc6 medium (glucose concentration: 20 g/l; xylose concentration: 60 g/l; yeast extract concentration: 10 g/l; and acetic acid concentration: 6 g/l) (concentration: 0.3 g dry cells/l), and the fermentation test was carried out via agitation culture at 80 rpm with an amplitude of 35 mm at 30° C. A rubber stopper into which a needle (i.d.: 1.5 mm) has been inserted was used to cap each flask, and a check valve was mounted on the tip of the needle to maintain the anaerobic conditions in the flask.

Sampling was carried out 65 hours after the initiation of fermentation, and glucose, xylose, acetic acid, and ethanol in the fermentation liquor were assayed via HPLC (LC-10A; Shimadzu Corporation) under the conditions described below.

### Column: Aminex HPX-87H

Mobile phase: 0.01N H₂SO₄
Flow rate: 0.6 ml/min

### Temperature: 30° C.

Detection apparatus: Differential refractometer (RID-10A)

### <Results of Fermentation Test>

The results of the fermentation test are shown in Table 1.

TABLE 1

| | | Ethanol | Xylose | Glucose | Acetic acid |
|---|---|---|---|---|---|
| Strain | Genotype | (g/l) | (g/l) | (g/l) | (g/l) |
| Uz928 | mhpF | 25.9 | 23.1 | 0.0 | 5.86 |
| Uz926 | mhpF [symbol] adh2 | 22.0 | 26.5 | 0.0 | 5.86 |
| Uz1012 | mhpF ADH1 | 24.0 | 23.2 | 0.0 | 5.92 |
| Uz736 | mhpF [symbol] adh2 | 29.0 | 2.5 | 0.0 | 5.42 |
| | ADH1 | | | | |
| Uz1048 | [symbol] adh2 | 20.6 | 30.0 | 0.0 | 5.62 |
| Uz1047 | ADH1 | 21.0 | 30.6 | 0.0 | 5.85 |
| Uz1049 | Cont. | 22.7 | 25.0 | 0.0 | 5.90 |

As is apparent from Table 1, the rate of xylose assimilation and the ethanol productivity of the Uz736 strains exhibiting mhpF and ADH1 gene overexpression and ADH2 gene disruption were remarkably improved, compared with the results for the mhpF-overexpressing strains. Since ADH2-disrupted strains and ADH1-overexpressing strains do not exhibit improved rates of xylose assimilation, overexpression and disruption as described above are considered to yield synergistic effects. In addition, the Uz736 strain was found to have improved acetic-acid-assimilating ability since acetic acid concentration in a medium was lowered to a significant degree.

### Example 2

In the present example, a recombinant yeast strain was prepared through introduction of a xylose isomerase gene and the mhpF gene of E. coli, the adhE gene, the acetaldehyde dehydrogenase gene derived from Clostridium beijerinckii, or the acetaldehyde dehydrogenase gene derived from Chlamydomonas reinhardtii. Either or both of a pair of endogenous ADH2 genes were disrupted in recombinant yeast prepared in the present Example.

### <Production of Vectors for Gene Introduction>(1) Plasmid for XI, XKS1, TKL1, TAL1, RKI1, and RPE1 Gene Introduction and GRE3 Gene Disruption

A plasmid (pUC-5U_GRE3-P_HOR7-TKL1-TAL1-FBA1_P-P_ADH1-RPE1-RKI1-TEF1_P-P_TDH1-XI_N337C-T_DIT1-P_TDH3-XKS1-T_HIS3-LoxP-G418-LoxP-3U_GRE3) was prepared. This plasmid comprises, at the GRE3 gene locus, a sequence necessary for GRE3 gene disruption and introduction of the following genes into yeast: a mutated gene for which the rate of xylose assimilation has been improved as a result of substitution of asparagine at amino acid position 377 of the xylose isomerase gene derived from the intestinal protozoa of Reticulitermes speratus with cysteine (XI_N337C); a yeast-derived xylulokinase (XKS1) gene; a transketolase 1 (TKL1) gene of the pentose phosphate pathway; a transaldolase 1 (TALI) gene; a ribulose phosphate epimerase 1 (RPE1) gene; and a ribose phosphate ketoisomerase (RKI1) gene.

The construction of the plasmid comprises: the TKL1 gene derived from the Saccharomyces cerevisiae BY4742 strain in which an HOR7 promoter is added on the 5′ side; the TALI gene in which an FBA1 promoter is added; the RKI1 gene in which an ADH1 promoter is added; the RPE1 gene in which a TEF1 promoter is added; XI_N337C in which a TDH1 promoter and a DIT1 terminator are added (prepared through the total synthesis on the basis of a sequence designed by changing codons over the entire region in accordance with the frequency of codon usage of the yeast strain); the XKS1 gene in which a TDH3 promoter and an HIS3 terminator are added; a gene sequence (GRE3U) comprising an upstream region of approximately 700 by from the 5′ terminus of the GRE3 gene and a DNA sequence (GRE3D) comprising a downstream region of approximately 800 by from the 3′ terminus of the GRE3 gene, which are regions to be integrated into the yeast genome via homologous recombination; and a gene sequence (G418 marker) comprising the G418 gene, which is a marker. The LoxP sequence was introduced on the both sides of the marker gene, thereby making it possible to remove the marker.

In addition, each DNA sequence contained in the plasmid can be amplified using primers listed in table 2. In order to ligate DNA fragments, a desired plasmid to be obtained as a final product was prepared in the following manner. A DNA sequence was added to each primer listed in table 2 such that the DNA sequence overlapped its adjacent DNA sequence by approximately 15 bp. The primers were used to amplify desired DNA fragments using, as templates, Saccharomyces cerevisiae BY4742 genome, DNA of the XI_N337C-synthesizing gene, and synthetic DNA of the LoxP sequence. The DNA fragments were sequentially ligated using an In-Fusion HD Cloning Kit (Takara Bio Inc.) or the like, followed by cloning into plasmid pUC19.

TABLE 2

| Amplified DNA | | SEQ |
|---|---|---|
| fragment | Primer sequence | ID NO: |
| GRE3U | 5′-TGGGAATATTACCGCTCGA | 25 |
| | AG-3′ | |
| | 5′-CTTTAAAAAATTTCCAATT | 26 |
| | TTCCTTTACG-3′ | |
| HOR7 promoter | 5′-TCGCAGCCACGGGTCAA | 27 |
| | C-3′ | |
| | 5′-TTTTATTATTAGTCTTTTT | 28 |
| | TTTTTTTGACAATATCTG-3′ | |
| TKL1 | 5′-ATGACTCAATTCACTGACA | 29 |
| (including the | TTGATAAGCTAG-3′ | |
| terminator | 5′-ATATTCTTTATTGGCTTTA | 30 |
| region) | TACTTGAATGGTG-3′ | |
| TAL1 | 5′-GACGTTGATTTAAGGTGGT | 31 |
| (including the | TCCGG-3′ | |
| terminator | 5′-ATGTCTGAACCAGCTCAAA | 32 |
| region) | AGAAAC-3′ | |
| FBA1 promoter | 5′-TTTGAATATGTATTACTTG | 33 |
| | GTTATGGTTATATATGAC-3′ | |
| | 5′-ACTGGTAGAGAGCGACTTT | 34 |
| | GTATGC-3′ | |
| ADH1 promoter | 5′-GCTTTCAATTCATTTGGGT | 35 |
| | GTG-3′ | |
| | 5′-TGTATATGAGATAGTTGAT | 36 |
| | TGTATGCTTGG-3′ | |
| RPE1 | 5′-ATGGTCAAACCAATTATAG | 37 |
| (including the | CTCCCAGTA-3′ | |
| terminator | 5′-AAATGGATATTGATCTAGA | 38 |
| region) | TGGCGG-3′ | |
| RKI1 | 5′-CTTGGTGTGTCATCGGTAG | 39 |
| (including the | TAACG-3′ | |
| terminator | 5′-ATGGCTGCCGGTGTCC | 40 |
| region) | C-3′ | |
| TEF1 promoter | 5′-TTGTAATTAAAACTTAGAT | 41 |
| | TAGATTGCTATGCTTTC-3′ | |
| | 5′-AGGAACAGCCGTCAAGG | 42 |
| | G-3′ | |
| TDH1 promoter | 5′-CTTCCCTTTTACAGTGCTT | 43 |
| | CGGAAAAGC-3′ | |
| | 5′-TTTGTTTTGTGTGTAAATT | 44 |
| | TAGTGAAGTACTG-3′ | |
| XI_N337C | 5′-ATGTCTCAAATTTTTAAGG | 45 |
| | ATATCCCAG-3′ | |
| | 5′-TTATTGAAACAAAATTTGG | 46 |
| | TTAATAATAC-3′ | |
| DIT1 terminator | 5′-TAAAGTAAGAGCGCTACAT | 47 |
| | TGGTCTACC-3′ | |
| | 5′-TTACTCCGCAACGCTTTTC | 48 |
| | TGAAC-3′ | |
| TDH3 promoter | 5′-TAGCGTTGAATGTTAGCGT | 49 |
| | CAACAAC-3′ | |
| | 5′-TTTGTTTGTTTATGTGTGT | 50 |
| | TTATTCGAAACTAAGTTCTTG | |
| | G-3′ | |
| XKS1 | 5′-ATGTTGTGTTCAGTAATTC | 51 |
| | AGAGACAG-3′ | |
| | 5′-TTAGATGAGAGTCTTTTCC | 52 |
| | AGTTCGC-3′ | |
| HIS3 terminator | 5′-TGACACCGATTATTTAAAG | 53 |
| | CTGCAG-3′ | |
| | 5′-AGAGCGCGCCTCGTTCA | 54 |
| | G-3′ | |
| LoxP | 5′-AATTCCGCTGTATAGCTCA | 55 |
| (including a | TATCTTTC-3′ | |
| linker sequence) | 5′-AACGAGGCGCGCTCTAATT | 56 |
| | CCGCTGTATAGCTCATATC | |
| | T-3′ | |
| CYC1 promoter | 5′-ACGACATCGTCGAATATGA | 57 |
| | TTCAG-3′ | |
| | 5′-TATTAATTTAGTGTGTGTA | 58 |
| | TTTGTGTTTGTGTG-3′ | |
| G418 | 5′-ATGAGCCATATTCAACGGG | 59 |
| | AAAC-3′ | |
| | 5′-TTACAACCAATTAACCAAT | 60 |
| | TCTGATTAG-3′ | |
| URA3 terminator | 5′-TGCATGTCTACTAAACTCA | 61 |
| | CAAATTAGAGCTTCAATT-3′ | |
| | 5′-GGGTAATAACTGATATAAT | 62 |
| | TAAATTGAAGCTCTAATTT | |
| | G-3′ | |
| LoxP | 5′-CCCATAACTTCGTATAGCA | 63 |
| (including a | TACATTATACGAAGTTATTGAC | |
| linker sequence) | ACCGATTATTTAAAGCTG-3′ | |
| | 5′-GTATGCTGCAGCTTTAAAT | 64 |
| | AATCGG-3′ | |
| GRE3D | 5′-TCCAGCCAGTAAAATCCAT | 65 |
| | ACTCAAC-3′ | |
| | 5′-AAGGGGGAAGGTGTGGAAT | 66 |
| | C-3′ | |

(2) Plasmid for mhpF and ADH1 Gene Introduction and ADH2 Gene Disruption

A plasmid (pUC-5U_ADH2-P_TDH3-ADH1-T_ADH1-DIT1_T-mhpF-HOR7_P-URA3-3U_ADH2) was prepared. This plasmid comprises, at the ADH2 gene locus, a sequence necessary for ADH2 gene disruption and introduction of the acetaldehyde dehydrogenase gene (mhpF) derived from E. coli and the alcohol dehydrogenase 1 (ADH1) gene derived from yeast into yeast.

The construction of the plasmid comprises: the ADH1 gene derived from the Saccharomyces cerevisiae BY4742 strain in which a TDH3 promoter is added on the 5′ side; the mhpF gene in which an HOR7 promoter and a DIT1 terminator are added (prepared through the total synthesis on the basis of a sequence designed by changing codons over the entire region in accordance with the frequency of codon usage of the yeast strain); a gene sequence (ADH2U) comprising an upstream region of approximately 700 by from the 5′ terminus of the ADH2 gene and a DNA sequence (ADH2D) comprising a downstream region of approximately 800 by from the 3′ terminus of the ADH2 gene, which are regions to be integrated into the yeast genome via homologous recombination; and a gene sequence (URA3 marker) comprising the URA3 gene, which is a marker.

In addition, each DNA sequence contained in the plasmid can be amplified using primers listed in table 3. In order to ligate DNA fragments, a desired plasmid to be obtained as a final product was prepared in the following manner. A DNA sequence was added to each primer listed in table 3 such that the DNA sequence overlapped its adjacent DNA sequence by approximately 15 bp. The primers were used to amplify desired DNA fragments using, as a template, Saccharomyces cerevisiae BY4742 genome or DNA of the mhpF-synthesizing gene. The DNA fragments were sequentially ligated using an In-Fusion HD Cloning Kit or the like, followed by cloning into plasmid pUC19.

TABLE 3

| Amplified DNA | | SEQ |
|---|---|---|
| fragment | Primer sequence | ID NO: |
| ADH2U | 5′-CTATGGGACTTCCGGGAA | 67 |
| | AC-3′ | |
| | 5′-TGTGTATTACGATATAGT | 68 |
| | TAATAGTTGATAGTTGATT | |
| | G-3′ | |
| TDH3 promoter | 5′-TAGCGTTGAATGTTAGCG | 69 |
| | TCAACAAC-3′ | |
| | 5′-TTTGTTTGTTTATGTGTG | 70 |
| | TTTATTCGAAACTAAGTTCTT | |
| | GG-3′ | |
| ADH1 | 5′-ATGTCTATCCCAGAAACT | 71 |
| (including the | CAAAAAGGTG-3′ | |
| terminator region) | 5′-TTGTCCTCTGAGGACATA | 72 |
| | AAATACACAC-3′ | |
| DIT1 terminator | 5′-TTACTCCGCAACGCTTTT | 73 |
| | CTGAAC-3′ | |
| | 5′-TAAAGTAAGAGCGCTACA | 74 |
| | TTGGTCTACC-3′ | |
| mhpF | 5′-TTATGCGGCCTCTCCTG | 75 |
| | C-3′ | |
| | 5′-ATGTCAAAGAGAAAAGTT | 76 |
| | GCTATTATCG-3′ | |
| HOR7 promoter | 5′-TTTTATTATTAGTCTTTT | 77 |
| | TTTTTTTTGACAATATCT | |
| | G-3′ | |
| | 5′-TCGCTCGCAGCCACGGG | 78 |
| | T-3′ | |
| URA3 (including | 5′-GATTCGGTAATCTCCGAG | 79 |
| the promoter and | CAG-3′ | |
| terminator regions) | 5′-GGGTAATAACTGATATAA | 80 |
| | TTAAATTGAAGCTCTAATTT | |
| | G-3′ | |
| ADH2D | 5′-GCGGATCTCTTATGTCTT | 81 |
| | TACGATTTATAGTTTTC-3′ | |
| | 5′-GAGGGTTGGGCATTCATC | 82 |
| | AG-3′ | |

(3) Plasmid for adhE and ADH1 Gene Introduction and ADH2 Gene Disruption

A plasmid (pUC-5U_ADH2-P_TDH3-ADH1-T_ADH1-DIT1_T-adhE-HOR7_P-URA3-3U_ADH2) was prepared. This plasmid comprises, at the ADH2 gene locus, a sequence necessary for ADH2 gene disruption and introduction of the acetaldehyde dehydrogenase gene (adhE) derived from E. coli and the alcohol dehydrogenase 1 (ADH1) gene derived from yeast into yeast.

The construction of the plasmid comprises: the ADH1 gene derived from the Saccharomyces cerevisiae BY4742 strain in which a TDH3 promoter is added on the 5′ side; the adhE gene in which an HOR7 promoter and a DIT1 terminator are added (NCBI accession No. NP_{—}415757.1; prepared through the total synthesis on the basis of a sequence designed by changing codons over the entire region in accordance with the frequency of codon usage of the yeast strain); a gene sequence (ADH2U) comprising an upstream region of approximately 700 by from the 5′ terminus of the ADH2 gene and a DNA sequence (ADH2D) comprising a downstream region of approximately 800 by from the 3′ terminus of the ADH2 gene, which are regions to be integrated into the yeast genome via homologous recombination; and a gene sequence (URA3 marker) comprising the URA3 gene, which is a marker.

In addition, each DNA sequence contained in the plasmid can be amplified using primers listed in table 4. In order to ligate DNA fragments, a desired plasmid to be obtained as a final product was prepared in the following manner. A DNA sequence was added to each primer listed in table 4 such that the DNA sequence overlapped its adjacent DNA sequence by approximately 15 bp. The primers were used to amplify desired DNA fragments using, as a template, a plasmid (pUC-5U_ADH2-P_TDH3-ADH1-T_ADH1-DIT1_T-mhpF-HOR7_P-URA3-3U_ADH2) or DNA of the adhE-synthesizing gene. The DNA fragments were sequentially ligated using an In-Fusion HD Cloning Kit or the like, followed by cloning into plasmid pUC19.

TABLE 4

| Amplified DNA | | SEQ |
|---|---|---|
| fragment | Primer sequence | ID NO: |
| Sequence other | 5′-TTTTATTATTAGTCTTTTT | 83 |
| than adhE | TTTTTTTGACAATATCTG-3′ | |
| | 5′-TAAAGTAAGAGCGCTACAT | 84 |
| | TGGTCTACC-3′ | |
| adhE | 5′-TTAAGCTGATTTCTTTGCT | 85 |
| | TTCTTCTCG-3′ | |
| | 5′-ATGGCAGTTACGAACGTTG | 86 |
| | CAGAG-3′ | |

(4) Plasmid for ADH2 Gene Disruption and Introduction of the Acetaldehyde Dehydrogenase Gene Derived from Clostridium beijerinckii and the ADH1 Gene

A plasmid (pUC-5U_ADH2-P_TDH3-ADH1-T_ADH1-DIT1_T-CloADH-HOR7_P-URA3-3U_ADH2) was prepared. This plasmid comprises, at the ADH2 gene locus, a sequence necessary for ADH2 gene disruption and introduction of the acetaldehyde dehydrogenase gene derived from Clostridium beijerinckii and the alcohol dehydrogenase 1 (ADH1) gene derived from yeast into yeast.

The construction of the plasmid comprises: the ADH1 gene derived from the Saccharomyces cerevisiae BY4742 strain in which a TDH3 promoter is added on the 5′ side; the acetaldehyde dehydrogenase gene derived from Clostridium beijerinckii in which an HOR7 promoter and a DIT1 terminator are added (NCBI accession No. YP_{—}001310903.1; prepared through the total synthesis on the basis of a sequence designed by changing codons over the entire region in accordance with the frequency of codon usage of the yeast strain); a gene sequence (ADH2U) comprising an upstream region of approximately 700 by from the 5′ terminus of the ADH2 gene and a DNA sequence (ADH2D) comprising a downstream region of approximately 800 by from the 3′ terminus of the ADH2 gene, which are regions to be integrated into the yeast genome via homologous recombination; and a gene sequence (URA3 marker) comprising the URA3 gene, which is a marker.

In addition, each DNA sequence contained in the plasmid can be amplified using primers listed in table 5. In order to ligate DNA fragments, a desired plasmid to be obtained as a final product was prepared in the following manner. A DNA sequence was added to each primer listed in table 5 such that the DNA sequence overlapped its adjacent DNA sequence by approximately 15 bp. The primers were used to amplify desired DNA fragments using, as a template, a plasmid (pUC-5U_ADH2-P_TDH3-ADH1-T_ADH1-DIT1_T-mhpF-HOR7_P-URA3-3U_ADH2) or DNA of the gene synthesizing acetaldehyde dehydrogenase derived from Clostridium beijerinckii. The DNA fragments were sequentially ligated using an In-Fusion HD Cloning Kit or the like, followed by cloning into plasmid pUC19.

TABLE 5

| Amplified DNA | | SEQ |
|---|---|---|
| fragment | Primer sequence | ID NO: |
| Sequence other | 5′-TTTTATTATTAGTCTTTTT | 87 |
| than CloADH | TTTTTTTGACAATATCTG-3′ | |
| | 5′-TAAAGTAAGAGCGCTACAT | 88 |
| | TGGTCTACC-3′ | |
| CloADH | 5′-TTAACCTGCTAAAACACAT | 89 |
| | CTTCTTTG-3′ | |
| | 5′-ATGAATAAGGATACCTTGA | 90 |
| | TTCCAACTAC-3′ | |

(5) Plasmid for ADH2 gene Disruption and Introduction of the Acetaldehyde Dehydrogenase Gene Derived from Chlamydomonas reinhardtii and the ADH1 Gene

A plasmid (pUC-5U_ADH2-P_TDH3-ADH1-T_ADH1-DIT1_T-Ch1aADH1-HOR7_P-UR A3-3U_ADH2) was prepared. This plasmid comprises, at the ADH2 gene locus, a sequence necessary for ADH2 gene disruption and introduction of the acetaldehyde dehydrogenase gene derived from Chlamydomonas reinhardtii and the alcohol dehydrogenase 1 (ADH1) gene derived from yeast into yeast.

The construction of the plasmid comprises: the ADH1 gene derived from the Saccharomyces cerevisiae BY4742 strain in which a TDH3 promoter is added on the 5′ side; the acetaldehyde dehydrogenase gene derived from Chlamydomonas reinhardtii in which an HOR7 promoter and a DIT1 terminator are added (NCBI accession No. 5729132; prepared through the total synthesis on the basis of a sequence designed by changing codons over the entire region in accordance with the frequency of codon usage of the yeast strain); a gene sequence (ADH2U) comprising an upstream region of approximately 700 by from the 5′ terminus of the ADH2 gene and a DNA sequence (ADH2D) comprising a downstream region of approximately 800 by from the 3′ terminus of the ADH2 gene, which are regions to be integrated into the yeast genome via homologous recombination; and a gene sequence (URA3 marker) comprising the URA3 gene, which is a marker.

In addition, each DNA sequence contained in the plasmid can be amplified using primers listed in table 6. In order to ligate DNA fragments, a desired plasmid to be obtained as a final product was prepared in the following manner. A DNA sequence was added to each primer listed in table 6 such that the DNA sequence overlapped its adjacent DNA sequence by approximately 15 bp. The primers were used to amplify desired DNA fragments using, as a template, a plasmid (pUC-5U_ADH2-P_TDH3-ADH1-T_ADH1-DIT1_T-mhpF-HOR7_P-URA3-3U_ADH2) or DNA of the gene synthesizing acetaldehyde dehydrogenase derived from Chlamydomonas reinhardtii. The DNA fragments were sequentially ligated using an In-Fusion HD Cloning Kit or the like, followed by cloning into plasmid pUC19.

TABLE 6

| Amplified DNA | | SEQ |
|---|---|---|
| fragment | Primer sequence | ID NO: |
| Sequence other | 5′-TTTTATTATTAGTCTTTTT | 91 |
| than ChlaADH1 | TTTTTTTGACAATATCTG-3′ | |
| | 5′-TAAAGTAAGAGCGCTACAT | 92 |
| | TGGTCTACC-3′ | |
| ChlaADH1 | 5′-TTAGTTGATTTTGGAGAAG | 93 |
| | AATTCAAGGG-3′ | |
| | 5′-ATGATGAGTTCCTCTCTGG | 94 |
| | TTAG-3′ | |

(6) Plasmid for mhpF Gene Introduction

A plasmid (pUC-ADH2-T_CYC1-DIT1_T-mhpF-HOR7_P-URA3-3U_ADH2) was prepared. This plasmid comprises, at the ADH2 gene locus, a sequence necessary for introduction of the acetaldehyde dehydrogenase gene (mhpF) derived from E. coli into yeast in the vicinity of the ADH2 gene locus without ADH2 gene disruption.

The construction of the plasmid comprises: the mhpF gene derived from the Saccharomyces cerevisiae BY4742 strain in which an HOR7 promoter and a DIT1 terminator are added on the 5′ side (prepared through the total synthesis on the basis of a sequence designed by changing codons over the entire region in accordance with the frequency of codon usage of the yeast strain); the ADH2 gene and a DNA sequence (ADH2D) comprising a downstream region of approximately 800 by from the 3′ terminus of the ADH2 gene, which are regions to be integrated into the yeast genome via homologous recombination; and a gene sequence (URA3 marker) comprising the URA3 gene, which is a marker.

In addition, each DNA sequence contained in the plasmid can be amplified using primers listed in table 7. In order to ligate DNA fragments, a desired plasmid to be obtained as a final product was prepared in the following manner. A DNA sequence was added to each primer listed in table 7 such that the DNA sequence overlapped its adjacent DNA sequence by approximately 15 bp. The primers were used to amplify desired DNA fragments using, as a template, a plasmid (pUC-5U_ADH2-P_TDH3-ADH1-T_ADH1-DIT1_T-mhpF-HOR7_P-URA3-3U_ADH2) or Saccharomyces cerevisiae BY4742 genome. The DNA fragments were sequentially ligated using an In-Fusion HD Cloning Kit or the like, followed by cloning into plasmid pUC19.

TABLE 7

| Amplified DNA | | SEQ ID |
|---|---|---|
| fragment | Primer sequence | NO: |
| ADH2 | 5′-GTCTGCCACACCGATT | 95 |
| | TGC-3′ | |
| | 5′-CTTATTTAGAAGTGTC | 96 |
| | AACAACGTATCTACC-3′ | |
| CYC1 | 5′-CTTAAGACAGGCCCCT | 97 |
| terminator | TTTCCTTTG-3′ | |
| | 5′-CTGCAGGAATTCGATA | 98 |
| | TCAAGCTTATC-3′ | |
| Sequence other | 5′-TTACTCCGCAACGCTT | 99 |
| than the above | TTCTGAAC-3′ | |
| | 5′-TCCCCGGGTACCGAGC | 100 |
| | TCG-3′ | |

### <Production of Yeast Strains Comprising Vectors Introduced Thereinto>

The diploid yeast strain, which is the Saccharomyces cerevisiae OC2 strain (NBRC2260), was selected in a 5-fluoroorotic acid-supplemented medium (Boeke, J. D., et al., 1987, Methods Enzymol., 154: 164-75.), and an uracil auxotrophic strain (OC2U) was designated as a host strain. The yeast strain was transformed using the Frozen-EZ Yeast Transformation II (ZYMO RESEARCH) in accordance with the protocols included thereinto.

The homologous recombination site of the plasmid prepared in (1) above (pUC-5U_GRE3-P_HOR7-TKL1-TAL1 -FBA1_P-P_ADH1-RPE1-RKI1-TEF1_P-P_TDH1-XI_N337C-T_DIT1-P_TDH3-XKS1-T_HIS3-LoxP-G418-LoxP-3U_GRE3) was amplified by PCR, the resulting amplified fragments were used to transform the OC2U strain, the resulting transformants were applied to YPD agar medium containing G418, and the grown colonies were then subjected to acclimatization. The acclimatized elite strain was designated as the Uz1252 strain. This strain was applied to sporulation medium (1% potassium phosphate, 0.1% yeast extract, 0.05% glucose, and 2% agar) for sporulation, and a diploid of the strain was formed by utilizing homothallism. The strain in which the mutated XI, TKL1, TAL1, RPE1, RKI1, and XKS1 genes had been incorporated into the GRE3 gene locus region of a diploid chromosome, and thus resulting in the disruption of the GRE3 gene, was obtained. The resulting strain was designated as the Uz1252-3 strain.

Subsequently, regions between homologous recombination sites of the plasmids pUC-5U_ADH2-P_TDH3-ADH1-T_ADH1-DIT1_T-mhpF-HOR7_P-URA3-3U_ADH2 prepared in (2) above, pUC-5U_ADH2-P_TDH3-ADH1-T_ADH1-DIT1_T-adhE-HOR7_P-URA3-3U_ADH2 prepared in (3) above, pUC-5U_ADH2-P_TDH3-ADH1-T_ADH1-DIT1_T-CloADH-HOR7_P-URA3-3U_ADH2 prepared in (4) above, pUC-5U_ADH2-P_TDH3-ADH1-T_ADH1-DIT1_T-Ch1aADH1-HOR7_P-URA 3-3U_ADH2 prepared in (5) above, and pUC-ADH2-T_CYC1-DIT1_T-mhpF-HOR7_P-URA3-3U_ADH2 prepared in (6) above were amplified by PCR, the resulting amplified fragments were used to transform the Uz1252-3 strain, the resulting transformants were applied to a uracil-free SD agar medium, and the grown colonies were then subjected acclimatization. The acclimatized elite strains were designated as the Uz1317 strain, the Uz1298 strain, the Uz1296 strain, the Uz1330 strain, and the Uz1320 strain.

Heterozygous recombination (1 copy) was observed in all of the above strains. Sporulation was induced in sporulation medium for the obtained Uz1317 strain, the Uz1298 strain, and the Uz1296 strain. The strains obtained through diploid formation by utilizing homothallism were designated as the Uz1319 strain, the Uz1318 strain, and the Uz1311 strain.

As a control, the uracil gene was amplified by PCR using the OC2 genome as a template, the resulting amplified fragments were used to transform the OC2U strain, the resulting transformants were applied to a uracil-free SD agar medium, and the grown colonies were then subjected to acclimatization. The obtained strain was designated as the Uz1313 strain. Sporulation was induced in sporulation medium for the obtained Uz1313 strain. The strain was subjected to diploid formation by utilizing homothallism. The resulting strain was designated as the Uz1323 strain.

Table 8 summarizes genotypes of the strains prepared in the Examples.

TABLE 8

| Strain | Genotype |
|---|---|
| Uz1317 | ADH2/adh2::mhpF ADH1 URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1319 | adh2::mhpF ADH1 URA3/adh2::mhpF ADH1 URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1298 | ADH2/adh2::adhE ADH1 URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1318 | adh2::adhE ADH1 URA3/adh2::adhE ADH1 URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1296 | ADH2/adh2:: CloADH ADH1 URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1311 | adh2:: CloADH ADH1 URA3/adh2:: CloADH ADH1 URA3 |
| | ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1330 | ADH2/adh2:: ChlaADH1 ADH1 URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1320 | ADH2/ADH2::mhpF URA3 ura3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1313 | URA3/ura3 |
| | gre3:: XI_N337C XKS1 TKL1 TAL1 RKI1 RPE1 G418/gre3:: |
| | XI_N337C XKS1 TKL1 TAL1 RKI1 RPE1 G418 |
| Uz1323 | URA3/URA3 |
| | gre3:: XI_N337C XKS1 TKL1 TAL1 RKI1 RPE1 G418/gre3:: |
| | XI_N337C XKS1 TKL1 TAL1 RKI1 RPE1 G418 |

### <Fermentation Test>

From among the strains obtained in the manner described above, two strains exhibiting high fermentation ability were selected and subjected to a fermentation test in flasks in the manner described below. The test strains were inoculated into 100-ml baffled flasks each comprising 20 ml of YPD liquid medium (yeast extract concentration: 10 g/l; peptone concentration: 20 g/l; and glucose concentration: 20 g/l), and culture was conducted at 30° C. and 120 rpm for 24 hours. The strains were harvested and inoculated into 10-ml flasks each comprising 8 ml of D60X80YPAc4 medium (glucose concentration: 60 g/l; xylose concentration: 80 g/l; yeast extract concentration: 10 g/l; peptone concentration: 20 g/l; and acetic acid concentration: 4 g/l) or D40X80YPAc2 medium (glucose concentration: 40 g/l; xylose concentration: 80 g/l; yeast extract concentration: 10 g/l; peptone concentration: 20 g/l; and acetic acid concentration: 2 g/l), and the fermentation test was carried out via agitation culture at 80 rpm with an amplitude of 35 mm at 30° C. A rubber stopper into which a needle (i.d.: 1.5 mm) has been inserted was used to cap each flask, and a check valve was mounted on the tip of the needle to maintain the anaerobic conditions in the flask.

Glucose, xylose, acetic acid, and ethanol in the fermentation liquor were assayed via HPLC (LC-10A; Shimadzu Corporation) under the conditions described below.

### Column: Aminex HPX-87H

Mobile phase: 0.01N H₂SO₄
Flow rate: 0.6 ml/min

### Temperature: 30° C.

Detection apparatus: Differential refractometer (RID-10A)

### <Results of Fermentation Test>

Tables 9 and 10 show the results of the fermentation test (concentration of prepared yeast: 0.3 g dry cells/l) for which D60X80YPAc4 medium was used and fermentation time was set to 66 hours. Tables 9 and 10 show the average values of data for the three recombinant strains, which had been independently obtained.

TABLE 9

| | Strain obtained through heterozygous introduction (1 copy) | | | | |
|---|---|---|---|---|---|
| | | Uz1320 | Uz1317 | Uz1298 | Uz1296 |
| | Uz1313 | ADH2::mhpF/ | adh2::mhpF | adh2::adhE | adh2::CloADH |
| | control | ADH2 | ADH1/ADH2 | ADH1/ADH2 | ADH1/ADH2 |
| Ethanol | 42.3 | 40.5 | 46.0 | 47.4 | 46.4 |
| concen- | | | | | |
| tration | | | | | |
| (g/l) | | | | | |
| Xylose | 29.9 | 39.5 | 26.7 | 20.7 | 24.2 |
| concen- | | | | | |
| tration | | | | | |
| (g/l) | | | | | |

TABLE 10

| | Strain obtained through homozygous introduction (2 copies) | | | |
|---|---|---|---|---|
| | | Uz1319 | Uz1318 | Uz1311 |
| | Uz1323 | adh2::mhpF | adh2::adhE | adh2::CloADH |
| | control | ADH1/ADH2 | ADH1/ADH2 | ADH1/ADH2 |
| Ethanol | 41.8 | 46.8 | 51.0 | 45.2 |
| concen- | | | | |
| tration | | | | |
| (g/l) | | | | |
| Xylose | 31.4 | 24.9 | 15.9 | 26.6 |
| concen- | | | | |
| tration | | | | |
| (g/l) | | | | |

Tables 11 and 12 show the results of the fermentation test (concentration of prepared yeast: 0.24 g dry cells/l) for which D40X80YPAc2 medium was used and fermentation time was set to 42 hours. In addition, table 13 shows the results of the fermentation test (concentration of prepared yeast: 0.3 g dry cells/l) for which D40X80YPAc2 medium was used and fermentation time was set to 42 hours for the strain obtained through heterozygous introduction. Tables 11 to 13 show the average values of data for the three recombinant strains, which had been independently obtained.

TABLE 11

| | Strain obtained through heterozygous introduction (1 copy) | | | | |
|---|---|---|---|---|---|
| | | Uz1320 | Uz1317 | Uz1298 | Uz1296 |
| | Uz1313 | ADH2::mhpF/ | adh2::mhpF | adh2::adhE | adh2::CloADH |
| | control | ADH2 | ADH1/ADH2 | ADH1/ADH2 | ADH1/ADH2 |
| Ethanol | 33.7 | 21.3 | 37.7 | 38.0 | 36.0 |
| concen- | | | | | |
| tration | | | | | |
| (g/l) | | | | | |
| Xylose | 33.3 | 60.3 | 26.9 | 26.3 | 30.6 |
| concen- | | | | | |
| tration | | | | | |
| (g/l) | | | | | |
| Acetic | 1.63 | 1.81 | 1.44 | 1.38 | 1.52 |
| acid | | | | | |
| concen- | | | | | |
| tration | | | | | |
| (g/l) | | | | | |

TABLE 12

| | Strain obtained through homozygous introduction (2 copies) | | | |
|---|---|---|---|---|
| | | Uz1319 | Uz1318 | Uz1311 |
| | Uz1323 | adh2::mhpF | adh2::adhE | adh2::CloADH |
| | control | ADH1/ADH2 | ADH1/ADH2 | ADH1/ADH2 |
| Ethanol | 34.8 | 37.1 | 37.8 | 36.0 |
| concen- | | | | |
| tration | | | | |
| (g/l) | | | | |
| Xylose | 28.8 | 26.5 | 25.8 | 27.9 |
| concen- | | | | |
| tration | | | | |
| (g/l) | | | | |
| Acetic | 1.57 | 1.27 | 1.16 | 1.46 |
| acid | | | | |
| concen- | | | | |
| tration | | | | |
| (g/l) | | | | |

TABLE 13

| | Strain obtained through heterozygous introduction (1 copy) | | | | | |
|---|---|---|---|---|---|---|
| | | | | | Uz1296 | Uz1330 |
| | | Uz1320 | Uz1317 | Uz1298 | adh2::Clo | adh2::Chla |
| | Uzl313 | ADH2::mhpF/ | adh2::mhpF | adh2::adhE | ADH | ADH1 |
| | control | ADH2 | ADH1/ADH2 | ADH1/ADH2 | ADH1/ADH2 | ADH1/ADH2 |
| Ethanol | 36.7 | 30.0 | 42.2 | 42.8 | 38.7 | 41.4 |
| concentration | | | | | | |
| (g/l) | | | | | | |
| Xylose | 28.7 | 43.6 | 15.8 | 25.5 | 25.5 | 21.0 |
| concentration | | | | | | |
| (g/l) | | | | | | |
| Acetic acid | 1.83 | 1.78 | 1.35 | 1.37 | 1.69 | 1.54 |
| concentration | | | | | | |
| (g/l) | | | | | | |

As is understood from tables 9-13, the rate of xylose assimilation significantly increased while the amount acetic acid obviously decreased for each strain, in which ADH2 was heterozygously or homozygously disrupted, and which overexpressed ADH1 and any one of the three forms of acetaldehyde dehydrogenase, compared with the control. As a result, ethanol productivity was improved. In addition, the amount of acetic acid in the strain obtained through homozygous introduction of the ADH2 gene decreased to a greater extent than that in the strain obtained through heterozygous introduction of the ADH2 gene. Meanwhile, in the case of the strain which expressed mhpF of acetaldehyde dehydrogenase alone, the rate of xylose assimilation decreased while the amount of acetic acid did not substantially decrease, resulting in no improvement in ethanol productivity.

All publications, patents, and patent applications cited herein are incorporated herein by reference in their entirety.

1. A method for producing ethanol comprising steps of culturing a recombinant yeast strain comprising a xylose isomerase gene and an acetaldehyde dehydrogenase gene introduced thereinto in a xylose-containing medium to perform ethanol fermentation.

2. The method for producing ethanol according to claim 1, wherein the xylose isomerase gene encodes the protein (a) or (b) below:
(a) a protein comprising the amino acid sequence as shown in SEQ ID NO: 4; or
(b) a protein comprising an amino acid sequence having 70% or higher identity with the amino acid sequence as shown in SEQ ID NO: 4 and having enzyme activity of converting xylose into xylulose.

3. The method for producing ethanol according to claim 1, wherein the acetaldehyde dehydrogenase gene encodes acetaldehyde dehydrogenase derived from E. coli.

4. The method for producing ethanol according to claim 3, wherein the acetaldehyde dehydrogenase derived from E. coli is the protein (a) or (b) below:
(a) a protein comprising the amino acid sequence as shown in SEQ ID NO: 2 or 20; or
(b) a protein comprising an amino acid sequence having 70% or higher identity with the amino acid sequence as shown in SEQ ID NO: 2 or 20 and having acetaldehyde dehydrogenase activity.

5. The method for producing ethanol according to claim 1, wherein the acetaldehyde dehydrogenase gene encodes acetaldehyde dehydrogenase derived from Clostridium beijerinckii.

6. The method for producing ethanol according to claim 5, wherein the acetaldehyde dehydrogenase derived from Clostridium beijerinckii is the protein (a) or (b) below:
(a) a protein comprising the amino acid sequence as shown in SEQ ID NO: 22; or
(b) a protein comprising an amino acid sequence having 70% or higher identity with the amino acid sequence as shown in SEQ ID NO: 22 and having acetaldehyde dehydrogenase activity.

7. The method for producing ethanol according to (1), wherein the acetaldehyde dehydrogenase gene encodes acetaldehyde dehydrogenase derived from Chlamydomonas reinhardtii.

8. The method for producing ethanol according to (5), wherein the acetaldehyde dehydrogenase derived from Chlamydomonas reinhardtii is the protein (a) or (b) below:
(a) a protein comprising the amino acid sequence as shown in SEQ ID NO: 24; or
(b) a protein comprising an amino acid sequence having 70% or higher identity with the amino acid sequence as shown in SEQ ID NO: 24 and having acetaldehyde dehydrogenase activity.

9. The method for producing ethanol according to claim 1, wherein the recombinant yeast strain further comprises the xylulokinase gene introduced thereinto.

10. The method for producing ethanol according to claim 1, wherein the recombinant yeast strain comprises a gene encoding an enzyme selected from the group of enzymes constituting a non-oxidative process in the pentose phosphate pathway.

11. The method for producing ethanol according to claim 10, wherein the group of enzymes constituting a non-oxidative process in the pentose phosphate pathway includes ribose-5-phosphate isomerase, ribulose-5-phosphate-3-epimerase, transketolase, and transaldolase.

12. The method for producing ethanol according to claim 1, wherein the medium contains cellulose and the ethanol fermentation proceeds simultaneously with saccharification by at least the cellulose.

13. The method for producing ethanol according to claim 1, wherein the recombinant yeast strain allows high-level expression of the alcohol dehydrogenase gene having activity of converting acetaldehyde into ethanol.

14. The method for producing ethanol according to claim 1, wherein the recombinant yeast strain shows a lowered expression level of the alcohol dehydrogenase gene having activity of converting ethanol into acetaldehyde.


SEQUENCE LISTING (100 sequences)

SEQ ID NO: 1 - DNA, 951 bp, Escherichia coli
  CDS (1)..(951)
atgtcaaagagaaaagttgctattatcggtagtggaaacatcggtactgatttgatgatcaagatccttagacatggacaacacttggaaatggctgttatggtcggtatcgatcctcagtcagacggacttgctagagccagaagaatgggtgttgctactacacatgaaggtgttattggattgatgaacatgccagagtttgcagatattgacatcgttttcgatgctacaagtgcaggtgctcacgttaagaatgacgctgccttgagagaagctaaacctgatattagattgatcgaccttaccccagcagctattggaccatactgtgttcctgttgtcaacttggaggccaatgttgatcaacttaacgttaatatggtcacatgcggtggacaggctaccattcctatggttgccgcagtctctagagttgctagagtccattatgccgaaattatcgcatccatcgcttcaaagagtgcaggtccaggaaccagagctaacattgacgaattcactgagaccacttctagagctatcgaagttgtcggtggagctgccaagggtaaagccattatcgttttgaatcctgcagagccacctcttatgatgagagatactgtttacgtcttgtctgacgaagcttcccaagatgacattgaggcctctatcaacgaaatggccgaggcagttcaggcttacgtcccaggttatagattgaagcaaagagttcagtttgaggtcattccacaagataaacctgttaatttgccaggtgtcggacagttctccggattgaaaacagctgtttggcttgaagtcgagggtgcagctcactacttgccagcttatgccggaaaccttgacattatgacttcttccgcattggctacagccgaaaagatggctcaatctcttgccagaaaagcaggagaggccgcataa

SEQ ID NO: 2 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 3 - DNA, 1320 bp, Intestinal Protist of Reticulitermes speratus
  CDS (1)..(1320)
atgtctcaaatttttaaggatatcccagttattaaatatgaaggtccagcttccaagaatcctttgagtttcaaatactacgatgcaaacaaggttattgatggtaaaccaatgaaggaacatttgagatacgcaatggcttggtggcataatttgtgtgctaccggtcaagatatgtttggtcctggtactgcagataaatccttcggtagtaagacagttggtaccatggaacatgcacatgctaaagttgatgctggttttgaattcatgtccaagttgggtgttgaatacttctgtttccatgatgctgatttggttccagaagcagatactttgagtgaaacaaacaaaagattggatgaaatcgctgaacatatcgttgctaagcaaaaggcaactggtattaaatgtttgtggggtacagcaaatttgttttctaaccctagattcttaaatggttctggttcttcaaactcagctgatgtttatgcatacgctgcagctcaaattaaaaaggctttggatttgactgttaaatttggtggtgttggttatgttttctggggtggtagagaaggttacgaaaccttgttgaacactgatgttaagttcgaacaagaaaacatcgctaacttgatgcatttggcagttacttacggtagatcaatcggttttaaaggtgacttctacattgaaccaaaacctaaggaaccaacaaagcatcaatatgattttgatgcagctactacaattggtttcattagacaatacggtttggaaaaggatttcaagttgaacatcgaagcaaaccatgctacattagcaggtcataccttccaacatgatttgagaatctctgctattaatggcatgttaggttcagttgatgcaaacacaggtgacccattgttaggttgggataccgatgaatttccttattccgtttacgataccactttggctatgtacgaaattattaaggcaggtggtttgaccggtggtttgaattttgattccaaggttagaagaccaagttacacacatgaagatttgttttacggtttcattttgggtatggattctttcgctttgggtttgattaaagcaaaggctttgattgcagatggtagattggattcattcgttaaggatagatacgcttcttacggttcaggtattggtgctaagattagagatcattctgcaactttggaagaattagcagcttatgcattagctaaagatacagttgctttgcctggttccggtagacaagaatacttagaaagtattattaaccaaattttgtttcaataa

SEQ ID NO: 4 - protein, 439 aa, Intestinal Protist of Reticulitermes speratus
MSQIFKDIPVIKYEGPASKNPLSFKYYDANKVIDGKPMKEHLRYAMAWWHNLCATGQDMFGPGTADKSFGSKTVGTMEHAHAKVDAGFEFMSKLGVEYFCFHDADLVPEADTLSETNKRLDEIAEHIVAKQKATGIKCLWGTANLFSNPRFLNGSGSSNSADVYAYAAAQIKKALDLTVKFGGVGYVFWGGREGYETLLNTDVKFEQENIANLMHLAVTYGRSIGFKGDFYIEPKPKEPTKHQYDFDAATTIGFIRQYGLEKDFKLNIEANHATLAGHTFQHDLRISAINGMLGSVDANTGDPLLGWDTDEFPYSVYDTTLAMYEIIKAGGLTGGLNFDSKVRRPSYTHEDLFYGFILGMDSFALGLIKAKALIADGRLDSFVKDRYASYGSGIGAKIRDHSATLEELAAYALAKDTVALPGSGRQEYLESIINQILFQ

SEQ ID NO: 5 - DNA, 1047 bp, Saccharomyces cerevisiae
  CDS (1)..(1047)
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 6 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 7 - DNA, 1047 bp, Saccharomyces cerevisiae
  CDS (1)..(1047)
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 8 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 9 - DNA, 1803 bp, Saccharomyces cerevisiae
  CDS (1)..(1803)
atgttgtgttcagtaattcagagacagacaagagaggtttccaacacaatgtctttagactcatactatcttgggtttgatctttcgacccaacaactgaaatgtctcgccattaaccaggacctaaaaattgtccattcagaaacagtggaatttgaaaaggatcttccgcattatcacacaaagaagggtgtctatatacacggcgacactatcgaatgtcccgtagccatgtggttagaggctctagatctggttctctcgaaatatcgcgaggctaaatttccattgaacaaagttatggccgtctcagggtcctgccagcagcacgggtctgtctactggtcctcccaagccgaatctctgttagagcaattgaataagaaaccggaaaaagatttattgcactacgtgagctctgtagcatttgcaaggcaaaccgcccccaattggcaagaccacagtactgcaaagcaatgtcaagagtttgaagagtgcataggtgggcctgaaaaaatggctcaattaacagggtccagagcccattttagatttactggtcctcaaattctgaaaattgcacaattagaaccagaagcttacgaaaaaacaaagaccatttctttagtgtctaattttttgacttctatcttagtgggccatcttgttgaattagaggaggcagatgcctgtggtatgaacctttatgatatacgtgaaagaaaattcagtgatgagctactacatctaattgatagttcttctaaggataaaactatcagacaaaaattaatgagagcacccatgaaaaatttgatagcgggtaccatctgtaaatattttattgagaagtacggtttcaatacaaactgcaaggtctctcccatgactggggataatttagccactatatgttctttacccctgcggaagaatgacgttctcgtttccctaggaacaagtactacagttcttctggtcaccgataagtatcacccctctccgaactatcatcttttcattcatccaactctgccaaaccattatatgggtatgatttgttattgtaatggttctttggcaagggagaggataagagacgagttaaacaaagaacgggaaaataattatgagaagactaacgattggactctttttaatcaagctgtgctagatgactcagaaagtagtgaaaatgaattaggtgtatattttcctctgggggagatcgttcctagcgtaaaagccataaacaaaagggttatcttcaatccaaaaacgggtatgattgaaagagaggtggccaagttcaaagacaagaggcacgatgccaaaaatattgtagaatcacaggctttaagttgcagggtaagaatatctcccctgctttcggattcaaacgcaagctcacaacagagactgaacgaagatacaatcgtgaagtttgattacgatgaatctccgctgcgggactacctaaataaaaggccagaaaggactttttttgtaggtggggcttctaaaaacgatgctattgtgaagaagtttgctcaagtcattggtgctacaaagggtaattttaggctagaaacaccaaactcatgtgcccttggtggttgttataaggccatgtggtcattgttatatgactctaataaaattgcagttccttttgataaatttctgaatgacaattttccatggcatgtaatggaaagcatatccgatgtggataatgaaaattgggatcgctataattccaagattgtccccttaagcgaactggaaaagactctcatctaa

SEQ ID NO: 10 - protein, 600 aa, Saccharomyces cerevisiae
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 11 - DNA, 1008 bp, Saccharomyces cerevisiae
  CDS (1)..(1008)
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 12 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 13 - DNA, 2043 bp, Saccharomyces cerevisiae
  CDS (1)..(2043)
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 14 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 15 - DNA, 717 bp, Saccharomyces cerevisiae
  CDS (1)..(717)
atggtcaaaccaattatagctcccagtatccttgcttctgacttcgccaacttgggttgcgaatgtcataaggtcatcaacgccggcgcagattggttacatatcgatgtcatggacggccattttgttccaaacattactctgggccaaccaattgttacctccctacgtcgttctgtgccacgccctggcgatgctagcaacacagaaaagaagcccactgcgttcttcgattgtcacatgatggttgaaaatcctgaaaaatgggtcgacgattttgctaaatgtggtgctgaccaatttacgttccactacgaggccacacaagaccctttgcatttagttaagttgattaagtctaagggcatcaaagctgcatgcgccatcaaacctggtacttctgttgacgttttatttgaactagctcctcatttggatatggctcttgttatgactgtggaacctgggtttggaggccaaaaattcatggaagacatgatgccaaaagtggaaactttgagagccaagttcccccatttgaatatccaagtcgatggtggtttgggcaaggagaccatcccgaaagccgccaaagccggtgccaacgttattgtcgctggtaccagtgttttcactgcagctgacccgcacgatgttatctccttcatgaaagaagaagtctcgaaggaattgcgttctagagatttgctagattag

SEQ ID NO: 16 - protein, 238 aa, Saccharomyces cerevisiae
MVKPIIAPSILASDFANLGCECHKVINAGADWLHIDVMDGHFVPNITLGQPIVTSLRRSVPRPGDASNTEKKPTAFFDCHMMVENPEKWVDDFAKCGADQFTFHYEATQDPLHLVKLIKSKGIKAACAIKPGTSVDVLFELAPHLDMALVMTVEPGFGGQKFMEDMMPKVETLRAKFPHLNIQVDGGLGKETIPKAAKAGANVIVAGTSVFTAADPHDVISFMKEEVSKELRSRDLLD

SEQ ID NO: 17 - DNA, 777 bp, Saccharomyces cerevisiae
  CDS (1)..(777)
atggctgccggtgtcccaaaaattgatgcgttagaatctttgggcaatcctttggaggatgccaagagagctgcagcatacagagcagttgatgaaaatttaaaatttgatgatcacaaaattattggaattggtagtggtagcacagtggtttatgttgccgaaagaattggacaatatttgcatgaccctaaattttatgaagtagcgtctaaattcatttgcattccaacaggattccaatcaagaaacttgattttggataacaagttgcaattaggctccattgaacagtatcctcgcattgatatagcgtttgacggtgctgatgaagtggatgagaatttacaattaattaaaggtggtggtgcttgtctatttcaagaaaaattggttagtactagtgctaaaaccttcattgtcgttgctgattcaagaaaaaagtcaccaaaacatttaggtaagaactggaggcaaggtgttcccattgaaattgtaccttcctcatacgtgagggtcaagaatgatctattagaacaattgcatgctgaaaaagttgacatcagacaaggaggttctgctaaagcaggtcctgttgtaactgacaataataacttcattatcgatgcggatttcggtgaaatttccgatccaagaaaattgcatagagaaatcaaactgttagtgggcgtggtggaaacaggtttattcatcgacaacgcttcaaaagcctacttcggtaattctgacggtagtgttgaagttaccgaaaagtga

SEQ ID NO: 18 - protein, 258 aa, Saccharomyces cerevisiae
MAAGVPKIDALESLGNPLEDAKRAAAYRAVDENLKFDDHKIIGIGSGSTVVYVAERIGQYLHDPKFYEVASKFICIPTGFQSRNLILDNKLQLGSIEQYPRIDIAFDGADEVDENLQLIKGGGACLFQEKLVSTSAKTFIVVADSRKKSPKHLGKNWRQGVPIEIVPSSYVRVKNDLLEQLHAEKVDIRQGGSAKAGPVVTDNNNFIIDADFGEISDPRKLHREIKLLVGVVETGLFIDNASKAYFGNSDGSVEVTEK

SEQ ID NO: 19 - DNA, 2676 bp, Escherichia coli
  CDS (1)..(2676)
atggcagttacgaacgttgcagagcttaatgcccttgtggagagggttaagaaagcacaaagggaatacgccagcttcacgcaagaacaagttgacaagattttcagggcagccgcgctagcggcagctgatgcgagaataccccttgctaagatggcagtcgcagaaagcggaatgggaattgtggaagataaggtcatcaagaatcatttcgcttctgagtatatctacaacgcgtataaggatgagaagacttgcggcgtcttatctgaggacgatacattcggcacaataactatcgctgaacctatagggatcatctgtggtatagttccaactacgaaccctacctcaactgctatattcaagagtcttatttctttgaaaacccgtaacgctatcatcttttctccacatccaagagccaaggacgccaccaacaaggcggcggacatcgtgctgcaggcggccatcgcggcgggcgcgcctaaggatctaattggctggatcgaccaacctagcgttgaactttccaatgctcttatgcaccatcctgatattaatttgatacttgcgactggaggccctgggatggtaaaggccgcttatagcagcggtaagcccgccatcggtgttggcgctggaaatacgccggtagtcattgatgagaccgccgacattaagagggcagtcgcgtcagttttgatgtctaagacttttgacaatggagtaatctgcgcgtcagagcagtccgttgtcgttgtggattcagtatacgacgcggtcagagaaaggttcgcgactcacggagggtacctacttcaagggaaggagttgaaagccgtccaggacgttatccttaagaatggtgcattgaacgccgccattgtaggccaaccagcgtataaaatagccgaacttgcgggctttagcgtaccagaaaacactaaaatacttattggtgaagtcaccgtggttgatgagtcagaacccttcgcccatgagaaactttccccaacactggcgatgtacagagcgaaggactttgaagatgcagtggaaaaagccgaaaagcttgttgcaatgggcggtatcggccacacaagctgcctttacacagatcaggataaccaaccagcaagagttagttatttcggccagaaaatgaagacggctaggatactaatcaacaccccggccagccaaggaggcattggtgacctgtataacttcaagttggctccttctcttacattgggctgtggttcctggggcggaaactctatctctgagaacgttggacctaagcatcttatcaataagaaaaccgtcgctaagcgtgctgagaacatgctttggcacaaactgccgaagtcaatctatttccgtaggggctccctgcctatcgcgttagatgaagtcatcaccgatgggcataagagagcactaatcgtcacagataggttcctattcaacaacggttacgcagaccaaatcacgtctgttctgaaagcggccggtgtcgaaacagaggtgttcttcgaagtggaagccgacccgacattaagtattgtcaggaaaggagctgagcttgcgaatagttttaagccggatgtcatcatcgctttgggaggaggatccccgatggatgctgctaagatcatgtgggtcatgtatgaacacccggagacacactttgaagagttggcgttgagattcatggatattaggaaaaggatctataaattccctaagatgggagtgaaagccaagatgatagcagtgacgaccaccagtggaaccgggagtgaagtgactccgtttgcggttgttactgacgacgctacgggccagaagtatcccctagccgactatgcattgacgccggatatggcgattgtggacgcgaacctggttatggatatgccaaagtcactttgcgcattcggtggacttgacgcagttacacatgcaatggaggcatatgtgtccgtgcttgcttcagagtttagcgatggacaggcacttcaagccttgaagttactaaaggaataccttcccgcatcttaccatgagggcagcaagaaccctgttgcccgtgagagagtacatagtgcggctaccattgccggtatcgcattcgctaatgctttcctgggtgtgtgtcattcaatggcccataagcttggatcccagtttcacatcccacacggattagccaacgctcttcttatatgtaacgtcatccgttataatgccaacgacaaccctacaaagcaaaccgcattctcacaatatgacaggccgcaggcgaggcgtcgttatgcagaaatagccgatcacctaggcttatccgcgccaggagatcgtacagctgctaagattgaaaagttgcttgcgtggttagaaacgcttaaggccgaactaggcattccaaagtctattagagaggcgggagttcaagaggcggatttccttgctaacgtggacaagcttagcgaagatgcattcgatgatcagtgcactggagccaacccgagatatcctctgatttcagaattgaagcaaattttacttgatacatactatgggagagactacgttgagggagagaccgcagcgaagaaggaggcggcgccggctaaggccgagaagaaagcaaagaaatcagcttaa

SEQ ID NO: 20 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 21 - DNA, 1407 bp, Clostridium beijerinckii
  CDS (1)..(1407)
atgaataaggataccttgattccaactacaaaggatttgaaggttaagacaaacggtgaaaacatcaacttgaaaaattacaaggataactcttcatgtttcggtgttttcgaaaacgttgaaaacgctatttccagtgctgttcatgcacaaaagattttgtcattgcattacactaaagaacaaagagaaaagattatcacagaaatcagaaaggctgcattgcaaaataaggaagttttggcaaccatgatcttggaagaaactcatatgggtagatacgaagataagatcttgaagcatgaattagttgctaaatacacaccaggtactgaagatttgaccactacagcatggtccggtgacaatggtttaactgttgttgaaatgagtccttatggtgttattggtgctattactccatctacaaatcctaccgaaactgttatttgtaactcaatcggtatgattgctgctggtaatgctgttgtttttaacggtcatccatgtgctaagaaatgtgttgctttcgcagttgaaatgatcaataaggctatcatctcttgtggtggtccagaaaatttggttaccactattaagaaccctactatggaatctttggatgcaattattaagcatccatcaattaagttgttatgtggtacaggtggtcctggtatggttaagaccttgttgaactctggtaaaaaggctattggtgctggtgcaggtaacccacctgttattgttgatgatacagctgatatcgaaaaggcaggtagatctatcatcgaaggttgttcattcgataacaacttgccttgtatcgctgaaaaggaagttttcgttttcgaaaacgttgcagatgatttgatctccaacatgttaaagaataacgctgttattattaatgaagatcaagtttcaaagttaattgatttggttttacaaaagaataacgaaactcaagaatacttcatcaataagaaatgggttggtaaagatgctaagttgttcttagatgaaatcgatgttgaatctccatcaaacgttaagtgtatcatctgtgaagttaatgcaaaccatccttttgttatgacagaattgatgatgccaatcttgcctatcgttagagttaaggatattgatgaagctattaaatacgctaagatcgcagaacaaaacagaaagcattccgcatacatctatagtaagaacatcgataatttgaacagatttgaaagagaaatcgatacaaccatcttcgttaaaaatgctaagtctttcgcaggtgttggttacgaagctgaaggttttactacattcaccattgcaggttccactggtgaaggtattacaagtgctagaaacttcactagacaaagaagatgtgttttagcaggttaa

SEQ ID NO: 22 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 23 - DNA, 2865 bp, Chlamydomonas reinhardtii
  CDS (1)..(2865)
atgatgagttcctctctggttagtggtaagagagttgccgtaccttctgccgcaaaaccgtgcgctgcagttcctctacctagggttgctggcagaagaacggctgcaagagttgtgtgcgaagcagcgccttcaggtgctgcccctgcctctccaaaagcagaggcagctgctcctgttgcagctgctcccgctacacctcatgcagaagttaagaaagaaagagcgccagcaactgacgaagcgttgacagagttaaaggctttgctaaagagagcacaaactgcacaagctcagtattctacatacactcaagaacaggtcgacgaaatctttcgtgcagccgctgaagcagctaacgcagccagaatacctttggccaaaatggcagtggaagaaactcgtatgggagtggctgaagataaagttgttaagaatcactttgcgtcagaattcatttataataagtataaacacacgaaaacttgtggtgtgatcgagcatgacccagcaggagggattcagaaagtggctgagcctgtaggtgtaatagctggtatcgttccaactactaatcccacttctacggcaatatttaagtccctactaagcttaaagaccagaaacgccttggttttgtgtccccaccctagggcagctaaaagtacaatcgcagctgccagaatagtgagagatgcggctgtcgcagctggagctcctcccaatataatttcttgggtggaaacaccgtcgttgccagtaagtcaagccttgatgcaagctactgaaattaacttgattttagctactggtggtcccgccatggtaagagctgcatacagttcagggaatccttctttgggagttggcgctgggaatacaccggctttgatagatgaaaccgcagatgtagccatggcagtctcttccattctactgagcaaaaccttcgacaatggtgtgatttgtgcttcagaacaaagcgttgtggtcgttgccaaggcttatgacgctgtcaggacagaattcgttaggagaggtgcctactttctaacggaagatgataaggtaaaggttagagcgggagtcgtagtagatggcaaattgaatccaaatatagttggccagtcaattccaaaacttgccgccctatttggcataaaggttcctcaaggcacaaaagttttgattggtgaggtagagaaaatcggtccagaagaagctttatctcaagagaaactttgccccatactggcaatgtatagagctccagattacgaccatggtgtcaaaatggcttgtgagttaataatgtacggtggtgccggacatacgagcgttttgtacaccaatccccttaataatgcgcacattcagcagtatcaatcagctgtcaaaacagtgagaatcttgattaacactccagctagtcaaggtgcaatcggtgatctttacaattttcatcttgatccctctcttacattgggctgtggtacatggggctcaacttctgtatcaacgaatgttggtccccaacatcttttgaacatcaaaaccgtaacagccaggagggaaaacatgttatggtttcgtgttccacctaaaatctactttaaaggtggatgtttagaagttgcgttaaccgacctgagagggaaaagcagagctttcattgtaactgataagccattatttgacatgggttatgctgataaagtcacgcacattttagactccattaacgtgcatcatcaagttttctaccacgttacaccggaccctacattggcttgcattgaagcggggttaaaagagatcttggaatttaaaccagacgttataatcgctttaggcggagggagtccaatggatgccgctaagatcatgtggctaatgtacgaatgcccagacaccagatttgatggactagccatgaggttcatggatattagaaagagagtttatgaggttccggaattgggcaagaaagcaaccatggtgtgcattcctacaacctctggtacaggttccgaagtcactccattttctgtagttacagatgaacgtttgggcgcaaaatatccactggcagattatgcattgactccgtcgatggccattgtcgatccacaattggtgttaaacatgcctaagaaattaaccgcctggggtggtattgacgcattgactcatgcgcttgagtcgtatgtctccatttgtgcgactgattatacgaagggattatcaagagaagccatatcactactgttcaagtacttgcccagggcatatgcaaacgggtcgaatgattacttagctagagaaaaggttcactatgccgcgactattgctggtatggcctttgcaaacgcattcttaggaatatgtcactccatggcgcataaattaggcgctgcttatcatgtgcctcatggtctagctaatgcagccttaatttcgcatgtcatacgttataacgctaccgatatgccagcgaaacaagccgctttcccacaatacgagtatcctactgcaaagcaagactatgctgatttggccaacatgctgggtcttggaggtaataccgtagatgagaaggtcatcaagttgattgaagcagtagaagaactgaaagctaaagttgacataccaccgactattaaggaaatctttaacgatccaaaggtggatgcggattttctggctaatgtcgacgctttggccgaggatgcatttgatgatcaatgtacaggagcaaatccaagatacccattgatggctgatttaaaacagttataccttgatgcccatgctgccccaattttaccagtcaaaacccttgaattcttctccaaaatcaactaa

SEQ ID NO: 24 - protein, 954 aa, Chlamydomonas reinhardtii
MMSSSLVSGKRVAVPSAAKPCAAVPLPRVAGRRTAARVVCEAAPSGAAPASPKAEAAAPVAAAPATPHAEVKKERAPATDEALTELKALLKRAQTAQAQYSTYTQEQVDEIFRAAAEAANAARIPLAKMAVEETRMGVAEDKVVKNHFASEFIYNKYKHTKTCGVIEHDPAGGIQKVAEPVGVIAGIVPTTNPTSTAIFKSLLSLKTRNALVLCPHPRAAKSTIAAARIVRDAAVAAGAPPNIISWVETPSLPVSQALMQATEINLILATGGPAMVRAAYSSGNPSLGVGAGNTPALIDETADVAMAVSSILLSKTFDNGVICASEQSVVVVAKAYDAVRTEFVRRGAYFLTEDDKVKVRAGVVVDGKLNPNIVGQSIPKLAALFGIKVPQGTKVLIGEVEKIGPEEALSQEKLCPILAMYRAPDYDHGVKMACELIMYGGAGHTSVLYTNPLNNAHIQQYQSAVKTVRILINTPASQGAIGDLYNFHLDPSLTLGCGTWGSTSVSTNVGPQHLLNIKTVTARRENMLWFRVPPKIYFKGGCLEVALTDLRGKSRAFIVTDKPLFDMGYADKVTHILDSINVHHQVFYHVTPDPTLACIEAGLKEILEFKPDVIIALGGGSPMDAAKIMWLMYECPDTRFDGLAMRFMDIRKRVYEVPELGKKATMVCIPTTSGTGSEVTPFSVVTDERLGAKYPLADYALTPSMAIVDPQLVLNMPKKLTAWGGIDALTHALESYVSICATDYTKGLSREAISLLFKYLPRAYANGSNDYLAREKVHYAATIAGMAFANAFLGICHSMAHKLGAAYHVPHGLANAALISHVIRYNATDMPAKQAAFPQYEYPTAKQDYADLANMLGLGGNTVDEKVIKLIEAVEELKAKVDIPPTIKEIFNDPKVDADFLANVDALAEDAFDDQCTGANPRYPLMADLKQLYLDAHAAPILPVKTLEFFSKIN

SEQ ID NO: 25 - DNA, 21 bp, Artificial Sequence
  primer
tgggaatattaccgctcgaag

SEQ ID NO: 26 - DNA, 29 bp, Artificial Sequence
  primer
ctttaaaaaatttccaattttcctttacg

SEQ ID NO: 27 - DNA, 18 bp, Artificial Sequence
  primer
tcgcagccacgggtcaac

SEQ ID NO: 28 - DNA, 37 bp, Artificial Sequence
  primer
ttttattattagtcttttttttttttgacaatatctg

SEQ ID NO: 29 - DNA, 31 bp, Artificial Sequence
  primer
atgactcaattcactgacattgataagctag

SEQ ID NO: 30 - DNA, 32 bp, Artificial Sequence
  primer
atattctttattggctttatacttgaatggtg

SEQ ID NO: 31 - DNA, 24 bp, Artificial Sequence
  primer
gacgttgatttaaggtggttccgg

SEQ ID NO: 32 - DNA, 25 bp, Artificial Sequence
  primer
atgtctgaaccagctcaaaagaaac

SEQ ID NO: 33 - DNA, 37 bp, Artificial Sequence
  primer
tttgaatatgtattacttggttatggttatatatgac

SEQ ID NO: 34 - DNA, 25 bp, Artificial Sequence
  primer
actggtagagagcgactttgtatgc

SEQ ID NO: 35 - DNA, 22 bp, Artificial Sequence
  primer
gctttcaattcatttgggtgtg

SEQ ID NO: 36 - DNA, 30 bp, Artificial Sequence
  primer
tgtatatgagatagttgattgtatgcttgg

SEQ ID NO: 37 - DNA, 28 bp, Artificial Sequence
  primer
atggtcaaaccaattatagctcccagta

SEQ ID NO: 38 - DNA, 25 bp, Artificial Sequence
  primer
aaatggatattgatctagatggcgg

SEQ ID NO: 39 - DNA, 24 bp, Artificial Sequence
  primer
cttggtgtgtcatcggtagtaacg

SEQ ID NO: 40 - DNA, 17 bp, Artificial Sequence
  primer
atggctgccggtgtccc

SEQ ID NO: 41 - DNA, 36 bp, Artificial Sequence
  primer
ttgtaattaaaacttagattagattgctatgctttc

SEQ ID NO: 42 - DNA, 18 bp, Artificial Sequence
  primer
aggaacagccgtcaaggg

SEQ ID NO: 43 - DNA, 28 bp, Artificial Sequence
  primer
cttcccttttacagtgcttcggaaaagc

SEQ ID NO: 44 - DNA, 32 bp, Artificial Sequence
  primer
tttgttttgtgtgtaaatttagtgaagtactg

SEQ ID NO: 45 - DNA, 28 bp, Artificial Sequence
  primer
atgtctcaaatttttaaggatatcccag

SEQ ID NO: 46 - DNA, 29 bp, Artificial Sequence
  primer
ttattgaaacaaaatttggttaataatac

SEQ ID NO: 47 - DNA, 28 bp, Artificial Sequence
  primer
taaagtaagagcgctacattggtctacc

SEQ ID NO: 48 - DNA, 24 bp, Artificial Sequence
  primer
ttactccgcaacgcttttctgaac

SEQ ID NO: 49 - DNA, 26 bp, Artificial Sequence
  primer
tagcgttgaatgttagcgtcaacaac

SEQ ID NO: 50 - DNA, 41 bp, Artificial Sequence
  primer
tttgtttgtttatgtgtgtttattcgaaactaagttcttgg

SEQ ID NO: 51 - DNA, 27 bp, Artificial Sequence
  primer
atgttgtgttcagtaattcagagacag

SEQ ID NO: 52 - DNA, 26 bp, Artificial Sequence
  primer
ttagatgagagtcttttccagttcgc

SEQ ID NO: 53 - DNA, 25 bp, Artificial Sequence
  primer
tgacaccgattatttaaagctgcag

SEQ ID NO: 54 - DNA, 18 bp, Artificial Sequence
  primer
agagcgcgcctcgttcag

SEQ ID NO: 55 - DNA, 27 bp, Artificial Sequence
  primer
aattccgctgtatagctcatatctttc

SEQ ID NO: 56 - DNA, 39 bp, Artificial Sequence
  primer
aacgaggcgcgctctaattccgctgtatagctcatatct

SEQ ID NO: 57 - DNA, 24 bp, Artificial Sequence
  primer
acgacatcgtcgaatatgattcag

SEQ ID NO: 58 - DNA, 33 bp, Artificial Sequence
  primer
tattaatttagtgtgtgtatttgtgtttgtgtg

SEQ ID NO: 59 - DNA, 23 bp, Artificial Sequence
  primer
atgagccatattcaacgggaaac

SEQ ID NO: 60 - DNA, 28 bp, Artificial Sequence
  primer
ttacaaccaattaaccaattctgattag

SEQ ID NO: 61 - DNA, 37 bp, Artificial Sequence
  primer
tgcatgtctactaaactcacaaattagagcttcaatt

SEQ ID NO: 62 - DNA, 39 bp, Artificial Sequence
  primer
gggtaataactgatataattaaattgaagctctaatttg

SEQ ID NO: 63 - DNA, 59 bp, Artificial Sequence
  primer
cccataacttcgtatagcatacattatacgaagttattgacaccgattatttaaagctg

SEQ ID NO: 64 - DNA, 25 bp, Artificial Sequence
  primer
gtatgctgcagctttaaataatcgg

SEQ ID NO: 65 - DNA, 26 bp, Artificial Sequence
  primer
tccagccagtaaaatccatactcaac

SEQ ID NO: 66 - DNA, 20 bp, Artificial Sequence
  primer
aagggggaaggtgtggaatc

SEQ ID NO: 67 - DNA, 20 bp, Artificial Sequence
  primer
ctatgggacttccgggaaac

SEQ ID NO: 68 - DNA, 38 bp, Artificial Sequence
  primer
tgtgtattacgatatagttaatagttgatagttgattg

SEQ ID NO: 69 - DNA, 26 bp, Artificial Sequence
  primer
tagcgttgaatgttagcgtcaacaac

SEQ ID NO: 70 - DNA, 41 bp, Artificial Sequence; identical to SEQ ID NO: 50
  primer

SEQ ID NO: 71 - DNA, 28 bp, Artificial Sequence
  primer
atgtctatcccagaaactcaaaaaggtg

SEQ ID NO: 72 - DNA, 28 bp, Artificial Sequence
  primer
ttgtcctctgaggacataaaatacacac

SEQ ID NO: 73 - DNA, 24 bp, Artificial Sequence
  primer
ttactccgcaacgcttttctgaac

SEQ ID NO: 74 - DNA, 28 bp, Artificial Sequence
  primer
taaagtaagagcgctacattggtctacc

SEQ ID NO: 75 - DNA, 18 bp, Artificial Sequence
  primer
ttatgcggcctctcctgc

SEQ ID NO: 76 - DNA, 28 bp, Artificial Sequence
  primer
atgtcaaagagaaaagttgctattatcg

SEQ ID NO: 77 - DNA, 37 bp, Artificial Sequence; identical to SEQ ID NO: 28
  primer

SEQ ID NO: 78 - DNA, 18 bp, Artificial Sequence
  primer
tcgctcgcagccacgggt

SEQ ID NO: 79 - DNA, 21 bp, Artificial Sequence
  primer
gattcggtaatctccgagcag

SEQ ID NO: 80 - DNA, 39 bp, Artificial Sequence; identical to SEQ ID NO: 62
  primer

SEQ ID NO: 81 - DNA, 35 bp, Artificial Sequence
  primer
gcggatctcttatgtctttacgatttatagttttc

SEQ ID NO: 82 - DNA, 20 bp, Artificial Sequence
  primer
gagggttgggcattcatcag

SEQ ID NO: 83 - DNA, 37 bp, Artificial Sequence; identical to SEQ ID NO: 28
  primer

SEQ ID NO: 84 - DNA, 28 bp, Artificial Sequence
  primer
taaagtaagagcgctacattggtctacc

SEQ ID NO: 85 - DNA, 28 bp, Artificial Sequence
  primer
ttaagctgatttctttgctttcttctcg

SEQ ID NO: 86 - DNA, 24 bp, Artificial Sequence
  primer
atggcagttacgaacgttgcagag

SEQ ID NO: 87 - DNA, 37 bp, Artificial Sequence; identical to SEQ ID NO: 28
  primer

SEQ ID NO: 88 - DNA, 28 bp, Artificial Sequence
  primer
taaagtaagagcgctacattggtctacc

SEQ ID NO: 89 - DNA, 27 bp, Artificial Sequence
  primer
ttaacctgctaaaacacatcttctttg

SEQ ID NO: 90 - DNA, 29 bp, Artificial Sequence
  primer
atgaataaggataccttgattccaactac

SEQ ID NO: 91 - DNA, 37 bp, Artificial Sequence; identical to SEQ ID NO: 28
  primer

SEQ ID NO: 92 - DNA, 28 bp, Artificial Sequence
  primer
taaagtaagagcgctacattggtctacc

SEQ ID NO: 93 - DNA, 29 bp, Artificial Sequence
  primer
ttagttgattttggagaagaattcaaggg

SEQ ID NO: 94 - DNA, 23 bp, Artificial Sequence
  primer
atgatgagttcctctctggttag

SEQ ID NO: 95 - DNA, 19 bp, Artificial Sequence
  primer
gtctgccacaccgatttgc

SEQ ID NO: 96 - DNA, 31 bp, Artificial Sequence
  primer
cttatttagaagtgtcaacaacgtatctacc

SEQ ID NO: 97 - DNA, 25 bp, Artificial Sequence
  primer
cttaagacaggccccttttcctttg

SEQ ID NO: 98 - DNA, 27 bp, Artificial Sequence
  primer
ctgcaggaattcgatatcaagcttatc

SEQ ID NO: 99 - DNA, 24 bp, Artificial Sequence
  primer
ttactccgcaacgcttttctgaac

SEQ ID NO: 100 - DNA, 19 bp, Artificial Sequence
  primer
tccccgggtaccgagctcg